(19) United States
(12) Patent Application Publication
(10) Pub. No.: US 2026/0066107 A1
(43) Pub. Date: Mar. 5, 2026

(54) PROGRAMMING CHANGE SIMULATION TO OPTIMIZE DEVICE RESOURCES

(71) Applicant: Cardiac Pacemakers, Inc., St. Paul, MN (US)

(72) Inventors: Kevin G. Wika, Blaine, MN (US); Jonathan H. Kelly, Woodbury, MN (US); Moira B. Sweeney, St. Paul, MN (US)

(21) Appl. No.: 19/304,813

(22) Filed: Aug. 20, 2025

Related U.S. Application Data
(60) Provisional application No. 63/689,966, filed on Sep. 3, 2024.

(51) Int. Cl. G16H 40/63

(52) U.S. Cl. CPC G16H 40/63

(57) ABSTRACT

Systems and methods are disclosed for evaluating patient condition and programming an ambulatory medical device to optimize resources of the ambulatory medical device, including updating a digital model of a patient based on received physiologic information of the patient, determining a change in the updated digital model of the patient, generating, based on the determined change in the updated digital model of the patient, a programming recommendation for the ambulatory medical device to optimize resources of the ambulatory medical device, and programming the ambulatory medical device based on the generated programming recommendation.

### CLAIM OF PRIORITY

This application claims the benefit of U.S. Provisional Application No. 63/689,966, filed on Sep. 3, 2024, which is hereby incorporated by reference in its entirety.

### TECHNICAL FIELD

This document relates generally to medical devices and more particularly to systems and methods for simulating ambulatory medical device programming changes and programming an ambulatory medical device to optimize ambulatory medical device resources.

### BACKGROUND

Ambulatory medical devices (AMDs), including implantable, subcutaneous, wearable, insertable, or one or more other medical devices, etc., can monitor, detect, or treat various conditions, including heart failure (HF), atrial fibrillation (AF), etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc. Frequent patient monitoring can provide early detection of worsening patient condition, including worsening heart failure or atrial fibrillation.

Ambulatory patient monitoring can provide early detection of worsening patient condition, including worsening heart failure or atrial fibrillation. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods are disclosed for evaluating patient condition and programming an ambulatory medical device to optimize resources of the ambulatory medical device, including updating a digital model of a patient based on received physiologic information of the patient, determining a change in the updated digital model of the patient, generating, based on the determined change in the updated digital model of the patient, a programming recommendation for the ambulatory medical device to optimize resources of the ambulatory medical device, and programming the ambulatory medical device based on the generated programming recommendation.

An example of subject matter (e.g., a system for evaluating patient condition and programming an ambulatory medical device to optimize resources of the ambulatory medical device) may comprise one or more processors and one or more memory devices storing instructions, which when executed by the one or more processors, cause the one or more processors to perform operations comprising receiving physiologic information of a patient from the ambulatory medical device, including physiologic information of the patient in a first time period generating a digital model of the patient based on the received physiologic information of the patient in the first time period receiving additional physiologic information of the patient from the ambulatory medical device subsequent to the first time period updating the digital model of the patient based on the received additional physiologic information of the patient subsequent to the first time period storing the updated digital model of the patient for use in evaluating programming changes for the ambulatory medical device determining a change in the updated digital model of the patient generating, based on the determined change in the updated digital model of the patient, a programming recommendation for the ambulatory medical device to optimize resources of the ambulatory medical device and programming the ambulatory medical device based on the generated programming recommendation to optimize resources of the ambulatory medical device.

In an example, which may be combined with any one or more examples described herein, the ambulatory medical device comprises an implantable medical device, the operations comprise receiving an indication of the time of implant of the implantable medical device, and the first time period comprises a post-implant time period including a pre-determined time period from the time of implant of the implantable medical device.

In an example, which may be combined with any one or more examples described herein, the ambulatory medical device of the patient is a first type of device, the operations comprise receiving physiologic information from a plurality of other patients have the first type of ambulatory medical device, and updating the digital model of the patient comprises continuously updating the digital model of the patient based on the received physiologic information from the plurality of other patients.

In an example, which may be combined with any one or more examples described herein, the operations comprise receiving parameter settings of the ambulatory medical device in the first time period and generating the digital model of the patient comprises based on the received physiologic information of the patient and the received parameter settings of the ambulatory medical device in first time period.

In an example, which may be combined with any one or more examples described herein, updating the digital model of the patient comprises continuously updating the digital model of the patient based on parameter settings of the ambulatory medical device.

In an example, which may be combined with any one or more examples described herein, generating the programming recommendation for the ambulatory medical device comprises generating a reprogramming recommendation to change at least one of a sensing, pacing, storage, communication, or therapy parameter of the ambulatory medical device to optimize resources of the ambulatory medical device.

In an example, which may be combined with any one or more examples described herein, the operations comprise generating, based on the generated programming recommendation, an alert to a user or process and providing an indication of the generated programming recommendation to the user or process and receiving an instruction to program the generated programming recommendation, wherein programming the ambulatory medical device based on the generated programming recommendation comprises programming the ambulatory medical device to implement the generated programming recommendation based on the received instruction to program the generated programming recommendation.

In an example, which may be combined with any one or more examples described herein, the operations comprise generating or accessing a digital model of the ambulatory medical device based on parameter settings of the ambulatory medical device and receiving a proposed programming change for the ambulatory medical device, the proposed programming change including at least one of a therapy change or a parameter setting change, wherein determining the change in the updated digital model of the patient comprises simulating application of the proposed programming change on the digital model of the patient using the digital model of the ambulatory medical device and predicting a change in the updated digital model of the patient of the proposed programming change based on training data from a plurality of other patients or ambulatory medical devices, wherein generating the programming recommendation for the ambulatory medical device comprises based on the predicted change in the updated digital model of the patient.

In an example, which may be combined with any one or more examples described herein, programming the ambulatory medical device based on the generated programming recommendation comprises programming the ambulatory medical device to implement the generated programming recommendation.

An example of subject matter (e.g., a system for predicting an outcome of programming changes to an ambulatory medical device and programming the ambulatory medical device to optimize resources of the ambulatory medical device) may comprise one or more processors and one or more memory devices storing instructions, which when executed by the one or more processors, cause the one or more processors to perform operations comprising receiving parameter settings of the ambulatory medical device generating a digital model of the ambulatory medical device based on the received parameter settings of the ambulatory medical device receiving a proposed programming change for the ambulatory medical device, the proposed programming change including at least one of a therapy change or a parameter setting change updating the digital model of the ambulatory medical device based on the proposed programming change, including simulating application of the proposed programming change on the digital model of the ambulatory medical device to predict an outcome of the proposed programming change based on training data from a plurality of other ambulatory medical devices determining a change in the updated digital model of the ambulatory medical device generating, based on the determined change in the updated digital model of the ambulatory medical device, a programming recommendation for the ambulatory medical device to optimize resources of the ambulatory medical device and programming the ambulatory medical device based on the generated programming recommendation to optimize resources of the ambulatory medical device.

In an example, which may be combined with any one or more examples described herein, the ambulatory medical device is a first type of device, the plurality of other ambulatory medical devices are the first type of device, and the operations comprise updating the digital model of the ambulatory medical device based on the received physiologic information from the plurality of other ambulatory medical devices.

In an example, which may be combined with any one or more examples described herein, the operations comprise generating or accessing a digital model of the patient based on physiologic information of the patient received from the ambulatory medical device and receiving a proposed programming change for the ambulatory medical device, the proposed programming change including at least one of a therapy change or a parameter setting change, wherein simulating application of the proposed programming change comprises simulating application of the proposed programming change on the digital model of the ambulatory medical device and the digital model of the patient to predict the outcome of the proposed programming change based on training data from a plurality of other ambulatory medical devices and a plurality of other patients, wherein updating the digital model of the ambulatory medical device comprises updating the digital model of the ambulatory medical device based on the predicted outcome of the proposed programming change, wherein the operations comprise updating the digital model of the patient based on the predicted outcome of the proposed programming change, wherein generating the programming recommendation for the ambulatory medical device comprises based on the predicted outcome of the proposed programming change.

In an example, which may be combined with any one or more examples described herein, simulating application of the proposed programming change comprises simulating application of a plurality of programming changes including the proposed programming change and predicting the outcome of the plurality of programming changes including the proposed programming change based on training data from a plurality of other patients or the training data from the plurality of other ambulatory medical devices.

In an example, which may be combined with any one or more examples described herein, determining the change in the updated digital model of the ambulatory medical device based on the predicted outcome of the proposed programming change.

An example of subject matter (e.g., a system for evaluating patient condition and programming an ambulatory medical device to optimize resources of the ambulatory medical device) may comprise one or more processors and one or more memory devices storing instructions, which when executed by the one or more processors, cause the one or more processors to perform operations comprising receiving physiologic information of a patient from the ambulatory medical device, including physiologic information of the patient in a first time period generating a digital model of the patient based on the received physiologic information of the patient in the first time period receiving additional physiologic information of the patient from the ambulatory medical device subsequent to the first time period determining a difference between the additional physiologic information subsequent to the first time period and the physiologic information from the first time period updating, based on the determined difference between the additional physiologic information subsequent to the first time period and the physiologic information from the first time period, the digital model of the patient using the received additional physiologic information of the patient subsequent to the first time period storing the updated digital model of the patient for use in evaluating programming changes for the ambulatory medical device determining a change in the updated digital model of the patient generating, based on the determined change in the updated digital model of the patient, a programming recommendation for the ambulatory medical device to optimize resources of the ambulatory medical device and programming the ambulatory medical device based on the generated programming recommendation to optimize resources of the ambulatory medical device.

In an example, which may be combined with any one or more examples described herein, the operations comprise generating a digital model of the ambulatory medical device based on received parameter settings of the ambulatory medical device and simulating application of the programming recommendation on both the digital model of the patient and the digital model of the ambulatory medical device to predict an outcome of the programming recommendation before programming the ambulatory medical device.

In an example, which may be combined with any one or more examples described herein, programming the ambulatory medical device to optimize resources of the ambulatory medical device comprises adjusting at least one of a sampling frequency of sensors, a data processing rate, a data storage rate, or a data transmission rate of the ambulatory medical device based on the determined changes in the continuously updated digital model of the patient.

An example of subject matter (e.g., a system for evaluating patient condition and programming an ambulatory medical device to optimize resources of the ambulatory medical device) may comprise one or more processors and one or more memory devices storing instructions, which when executed by the one or more processors, cause the one or more processors to perform operations comprising receiving physiologic information of a patient from the ambulatory medical device continuously updating a stored digital model of the patient based on the received physiologic information of the patient determining changes in the continuously updated digital model of the patient generating, based on the determined changes in the continuously updated digital model of the patient, a programming recommendation for the ambulatory medical device to optimize resources of the ambulatory medical device and programming the ambulatory medical device based on the generated programming recommendation to optimize resources of the ambulatory medical device.

In an example, which may be combined with any one or more examples described herein, the operations comprise receiving information of the ambulatory medical device continuously updating a stored digital model of the ambulatory medical device based on the received information of the ambulatory medical device determining changes in the continuously updated digital model of the ambulatory medical device, wherein generating the programming recommendation includes based on the determined changes in the continuously updated digital model of the patient and the determined changes in the continuously updated digital model of the ambulatory medical device.

In an example, which may be combined with any one or more examples described herein, programming the ambulatory medical device to optimize resources of the ambulatory medical device comprises adjusting at least one of a sampling frequency of sensors, a data processing rate, a data storage rate, or a data transmission rate of the ambulatory medical device based on the determined changes in the continuously updated digital model of the patient.

An example of subject matter (e.g., a method for evaluating patient condition and programming an ambulatory medical device to optimize resources of the ambulatory medical device) may comprise receiving physiologic information of a patient from the ambulatory medical device, including physiologic information of the patient in a first time period generating a digital model of the patient based on the received physiologic information of the patient in the first time period receiving additional physiologic information of the patient from the ambulatory medical device subsequent to the first time period updating the digital model of the patient based on the received additional physiologic information of the patient subsequent to the first time period storing the updated digital model of the patient for use in evaluating programming changes for the ambulatory medical device determining a change in the updated digital model of the patient generating, based on the determined change in the updated digital model of the patient, a programming recommendation for the ambulatory medical device to optimize resources of the ambulatory medical device and programming the ambulatory medical device based on the generated programming recommendation to optimize resources of the ambulatory medical device.

An example of subject matter (e.g., a method for predicting an outcome of programming changes to an ambulatory medical device and programming the ambulatory medical device to optimize resources of the ambulatory medical device) may comprise receiving parameter settings of the ambulatory medical device generating a digital model of the ambulatory medical device based on the received parameter settings of the ambulatory medical device receiving a proposed programming change for the ambulatory medical device, the proposed programming change including at least one of a therapy change or a parameter setting change updating the digital model of the ambulatory medical device based on the proposed programming change, including simulating application of the proposed programming change on the digital model of the ambulatory medical device to predict an outcome of the proposed programming change based on training data from a plurality of other ambulatory medical devices determining a change in the updated digital model of the ambulatory medical device generating, based on the determined change in the updated digital model of the ambulatory medical device, a programming recommendation for the ambulatory medical device to optimize resources of the ambulatory medical device and programming the ambulatory medical device based on the generated programming recommendation to optimize resources of the ambulatory medical device.

An example of subject matter (e.g., a method for evaluating patient condition and programming an ambulatory medical device to optimize resources of the ambulatory medical device) may comprise receiving physiologic information of a patient from the ambulatory medical device, including physiologic information of the patient in a first time period generating a digital model of the patient based on the received physiologic information of the patient in the first time period receiving additional physiologic information of the patient from the ambulatory medical device subsequent to the first time period determining a difference between the additional physiologic information subsequent to the first time period and the physiologic information from the first time period updating, based on the determined difference between the additional physiologic information subsequent to the first time period and the physiologic information from the first time period, the digital model of the patient using the received additional physiologic information of the patient subsequent to the first time period storing the updated digital model of the patient for use in evaluating programming changes for the ambulatory medical device determining a change in the updated digital model of the patient generating, based on the determined change in the updated digital model of the patient, a programming recommendation for the ambulatory medical device to optimize resources of the ambulatory medical device and programming the ambulatory medical device based on the generated programming recommendation to optimize resources of the ambulatory medical device.

An example of subject matter (e.g., a method for evaluating patient condition and programming an ambulatory medical device to optimize resources of the ambulatory medical device) may comprise receiving physiologic information of a patient from the ambulatory medical device continuously updating a stored digital model of the patient based on the received physiologic information of the patient determining changes in the continuously updated digital model of the patient generating, based on the determined changes in the continuously updated digital model of the patient, a programming recommendation for the ambulatory medical device to optimize resources of the ambulatory medical device and programming the ambulatory medical device based on the generated programming recommendation to optimize resources of the ambulatory medical device.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a table of parameter settings and a reduction of different combinations of parameter settings.

FIG. 2 illustrates a diagram of an example machine learning model.

FIG. 3 illustrates an example method.

FIG. 4 illustrates an example medical device system.

FIG. 5 illustrates an example patient management system.

FIG. 6 illustrates an example implantable medical device system.

FIGS. 7-9 illustrate example ambulatory medical devices systems, devices, and remote patient or programming systems.

FIG. 10 illustrates an example machine upon which any one or more of the techniques discussed herein may perform.

### DETAILED DESCRIPTION

Ambulatory medical devices are devices configured to be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) cardiac rhythm management devices configured to monitor or provide stimulation to the patient.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICDs), subcutaneous implantable cardioverter defibrillators (S-ICDs), cardiac resynchronization therapy defibrillators (CRT-Ds), insertable cardiac monitors (ICMs), leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (LV) (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Ambulatory medical devices can provide different monitoring, storage, communication, or therapy using different modes, however, with different power and resource requirements and varying effectiveness for different patients. For example, a variety of therapy modalities are available to patients, but not all patients receive the optimal medical device, therapy mode, or therapy parameter settings at first programming. Recent literature suggests that nearly 40% of current pacing therapy is suboptimal, where suboptimal is defined as cardiac resynchronization therapy resulting in cardiac capture of one or more chambers (typically the LV for CRT patients generally) in less than 98% of cardiac beats. One common reason for suboptimal cardiac resynchronization therapy is inappropriate programming of parameter settings of medical devices (e.g., implantable medical devices) configured to provide cardiac resynchronization therapy (e.g., CRT devices). Although discussed extensively herein with respect to cardiac capture, other desired clinical outcomes are contemplated, such as selection of pacing or sensing vectors, pacing mode, resource usage associated with communication or transmission of physiologic data from the ambulatory medical device or storage of physiologic data, etc.

The present inventors have recognized, among other things, systems and methods to evaluate and predict outcomes of programming changes for patients and ambulatory medical devices, and in certain examples make programming recommendations, using one or more digital models, such as a digital model of a patient, a digital model of an ambulatory medical device, or combinations thereof, to optimize resources of the ambulatory medical device, thereby extending a usable lifetime of the ambulatory medical device, such as with respect to utility provided to the patient prior to required replacement of the ambulatory medical device, etc. The one or more digital models (e.g., cloud-based digital models accessed, generated, or updated by the systems and methods described herein) can include a digital model of a particular patient, a digital model for a particular ambulatory medical device or particular type or configuration of a particular ambulatory medical device, or in certain examples combinations thereof. In this way, evaluation, prediction, and recommendations can be patient-centric (e.g., based first on a generated digital model of the patient), device-centric (e.g., based first on a generated digital model of an ambulatory medical device), or in other examples patient-and-device-centric (e.g., combinations of both, first one then the other, etc.).

In an example, the digital model of the patient can be generated for a particular patient based on physiologic information received from an ambulatory medical device implanted in, located on, or otherwise associated with the particular patient and be continuously updated as additional physiologic information is received, such as from the ambulatory medical device, from one or more other ambulatory medical devices associated with the particular patient, or in certain examples from determined indications, comorbidities, disease states, detected events, or information from medical records of the particular patient or received from the particular patient, such as patient demographics, etc.

In an example, the digital model of the ambulatory medical device can be generated for a respective ambulatory medical device based on information about the ambulatory medical device, such as based on a type, configuration, parameter settings, or in certain examples further with respect to physiologic information of one or more other patients having the type, configuration, or parameter settings of ambulatory medical device, etc.

Each of the one or more digital models has distinct advantages. For example, the digital model of the patient (e.g., a patient-centric model) can predict which of a plurality of different types of ambulatory medical device or configuration or parameter settings might provide the most benefit to the patient or a respective ambulatory medical device based on physiologic information from one or more other patients having similar physiologic information to the particular patient. In contrast, the digital model of the ambulatory medical device (e.g., a device-centric model) can predict which parameter setting or set of parameter settings might provide the most benefit to the particular patient or a respective ambulatory medical device given the type, configuration, or parameter settings of ambulatory medical device, resulting in a smaller amount of required computing resources (a smaller training set of data) or improved outcomes in situations where physiologic information from one or more other patients having similar physiologic information to the particular patient is lacking in the training data.

Additionally, concentrating on ambulatory medical device performance, for example, expected performance versus observed performance according to the digital model of the ambulatory medical device, enables monitoring and control of ambulatory device behavior and condition in a more focused and efficient way than through patient monitoring. Whereas device performance can be one aspect that results in changes to changes in a digital model of a patient, certain patients, in periods of good health, may tolerate or compensate for poor ambulatory medical device performance, but if patient condition worsens, the poor ambulatory medical device performance can accelerate patient decline in a time period where the patient may not be healthy enough to change the ambulatory medical device. Separately, concentrating on patient performance, for example, expected patient performance versus observed performance according to the digital model of the patient, maintains patient status and condition as a hallmark, separate from ambulatory medical device performance. Analyzing programming changes by simulating and predicting outcomes on the one or more digital models and comparing post-programming outcomes enables focused across-device or across-patient analysis at the areas where the machine learning model or training data is weakest, while also identifying beneficial programming changes across different patients or devices.

In an example, when a proposed programming change is received, such as a therapy modification or changes to parameter settings, the systems and methods can simulate application of the change on the one or more digital models to determine impact on the digital model of the patient or the digital model of the ambulatory medical device (or in certain examples combinations thereof), such as to predict or analyze the impact of the proposed programming change on the patient or the ambulatory medical device. By leveraging training data from a broad set of patients and devices, the systems and methods can predict the likely outcome of the proposed change and generate a recommendation regarding implementation, in certain examples confirming or denying a proposed programming change or in other examples generating one or more alternative programming changes providing more positive impact on the digital model of the patient, the digital model of the ambulatory medical device, or combinations thereof. Evaluating proposed programming changes by simulation on digital models improves safety and efficacy of device management while additionally sharing educational advances in programming and device management of physiologic conditions, further improving safety and efficacy of existing devices, reducing the likelihood of negative programming impacts to patients and resources associated with suboptimal parameter settings or device choices, and optimize resources of the ambulatory medical device.

The systems and methods described herein leverage a broad set of patient data and detected events to evaluate different types of programming changes for ambulatory medical devices, in certain examples in real-time and continuously after implementation. By simulating potential changes using the digital models before actually implementing them, the systems and methods can provide a comprehensive evaluation of the likely success of a programming change. This evaluation may include a percent confidence, a risk assessment (e.g., High/Medium/Low risk), and indications of potential concerns, issues, or edge cases that may arise.

The digital model of the ambulatory medical device can be created with a large amount of physiologic information from different input signals, such as cardiac electrical information, heart sounds, respiration, impedance pressure, activity, or other physiologic information or parameters of the patient. This large amount of physiologic information allows the system to evaluate simulated performance and produce more accurate outcome predictions based on previous implementation in similar situations, or implementations on either side of the specific proposed programming changes. For example, the systems and methods can simulate the effects of switching sensing vectors or changing sensing parameters based on device programming data.

In certain examples, the systems and methods can provide guidance on what changes should be made to achieve a desired clinical outcome. Clinicians can input clinical goals for the particular patient and the digital models, trained on a vast array of patient data and outcomes, can suggest specific programming changes to accomplish those goals. This “reverse engineering” approach allows clinicians to focus on the desired patient outcome rather than the technical details of device programming while also leveraging state-of-the-art literature and guidelines that may not yet be recognized by the clinician.

To ensure patient safety and maintain appropriate clinical oversight, the systems and methods can implement a two-factor approval process for accepting and applying suggested programming changes. This may require physician-level approval or a “two-key” system where two different users or components (e.g., the clinician and the recommendation system, the clinician and the user, such as through a user interface, etc.) must approve the change before it is applied to the patient device. This approach mirrors high-security systems that require multiple authorized individuals to access sensitive areas or information, providing both improved system safety and security in contrast to clinician-alone security and oversight. In this way, if proposed parameter settings are not predicted to improve patient condition, such as by more than an improvement threshold, or if other parameter settings are predicted to provide a greater improvement in patient condition, the proposed parameter settings can be omitted from implementation, or an alert or notification can be provided to the clinician or one or more other users.

As the systems and methods described herein continues to receive and analyze data from a growing patient population, its predictive capabilities and recommendations will become increasingly refined and accurate. The combination of continuously updated patient models, device simulations, and AI-driven recommendations has the potential to significantly improve the efficiency and effectiveness of implantable medical device management, ultimately leading to better patient outcomes and use of device resources, increasing effectiveness of devices and increasing device lifetime and the percentage of optimal impact on the patient over the limited lifespan of the device.

A cloud-based system, or a system having a cloud-based component, for predicting and evaluating programming changes to implantable medical devices offers several key advantages over traditional programming methods. For example, by leveraging vast amounts of patient data and sophisticated digital models, the system can provide more accurate and personalized recommendations for device optimization that provide substantive benefits to the device and patients associated therewith.

For example, the systems and methods described herein can create and maintain a digital “twin” of the particular patient (e.g., the digital model of the patient) and associated ambulatory medical device (e.g., the digital model of the ambulatory medical device). Such digital twins can include highly detailed virtual representation of the physiologic information of the particular patient and the associated ambulatory medical device, incorporating not only the device specifications and current settings but also simulated input signals that can mimic actual patient physiological data. By continuously updating these digital twins with real-time data from the patient and patient device, the systems and methods described herein can maintain an accurate model for simulation and prediction purposes, but also for interrogation by clinicians for reporting, education, or compliance, similar to a black box in an airplane.

In certain examples, the predictive capabilities can extend beyond simple parameter adjustments. By analyzing patterns in large datasets from multiple patients, the digital models can identify complex relationships between device settings, patient characteristics, and clinical outcomes. This allows the systems and methods described herein to suggest novel programming strategies that may not be immediately apparent to human clinicians, potentially leading to improved patient outcomes in challenging cases.

Further, as the patient model increases in complexity and nuance, less physiologic information may need to be communicated outside of an implantable medical device if the information is not adding to or deviating from the existing patient model-redundant information may not need to be recorded, stored, reported, or communicated out of the device using one or more communication circuits, which is commonly the largest source of power consumption in the ambulatory medica device, directly impacting device lifespan. In one example, storage or communication of physiologic data outside of an ambulatory or implantable medical device can be omitted if the physiologic information agrees with expected values. Moreover, storage and communication by the ambulatory medical device can be controlled by different parameter settings of the ambulatory medical device associated with each. For example, determinations, storage, recordings of high-resolution physiologic data (e.g., ECG or HS segments, etc.), or transmission of physiologic information (e.g., at a higher resolution or sampling frequency, such as by a loop recorder, etc.) can be omitted, such as by different parameter settings, if physiologic parameters are within expected values of the digital model. Once the physiologic information deviates from the digital model, such as by more than a deviation threshold, storage and transmission can be triggered, such as by adjusting one or more parameter settings, thereby optimizing device resources.

Additionally, security and data privacy are paramount in the design of remote or cloud-based systems. Patient data can be encrypted and anonymized to protect individual privacy while still allowing for meaningful analysis across patient populations. The two-factor approval process for implementing programming changes serves not only as a safety measure but also as an additional layer of security against unauthorized modifications to device settings.

The digital models described herein are designed to be continuously learning and improving. As more data is collected and more programming changes are evaluated, the predictive accuracy and the relevance of recommendations will increase. This creates a positive feedback loop, where better recommendations lead to improved patient outcomes, which in turn provide more valuable data for further refinement of the models.

Another innovative feature of the one or more digital models is the ability to perform “what-if” analyses with respect to the patient, the ambulatory medical device, or one or more other patients or ambulatory medical devices. Through interaction with the different models, clinicians can propose and explore multiple potential programming changes simultaneously, comparing predicted outcomes and risk assessments for each scenario. This allows for a more comprehensive evaluation of treatment options and can help clinicians make more informed decisions about device management and in certain examples the impact to the particular patient of different parameter settings, therapies, or even different ambulatory medical devices, which can be especially beneficial in determining which type of device to implant, which configuration to implement, or when to exchange or replace existing devices.

Cloud-based aspects of the system additionally facilitate collaboration and knowledge sharing among healthcare providers. Anonymized data and insights gained from successful programming strategies can be shared across institutions, potentially accelerating the adoption of best practices in device management and improving overall standards of care for patients with implantable medical devices.

In addition to its predictive capabilities, the system can also serve as a valuable tool for patient education and engagement. By providing clear visualizations of predicted outcomes and explaining the rationale behind recommended changes, the system can help patients better understand their treatment and potentially improve adherence to medical advice, improving device utilization and outcome and potentially reducing deliberation time by patients prior to implant, implementation, or adoption.

Further, in certain examples, instead of generating the digital model of the patient or the ambulatory medical device, the systems and methods herein can access a stored digital model for use in evaluating different ambulatory medical devices or programming changes for ambulatory medical devices, such as by simulating different parameter settings for one or more ambulatory medical devices on the digital model of the patient and updating the digital model of the patient, the ambulatory medical device, or combinations thereof prior to selecting, programming, or reprogramming an ambulatory medical device.

### Implant and Follow-Up

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

### Patient Response to Parameter Settings

In certain examples, multiple therapies or combinations of parameter settings can be evaluated for efficacy for a particular patient using physiologic information from the particular patient without requiring in-person appointments, including implementing parameter setting changes (e.g., including changes in modes) from a medical device system including a remote component separate from the ambulatory medical device. In an example, instead of each change in parameter settings requiring an in-person clinic visit or follow-up appointment, a physiologic status of the patient can be remotely monitored through the ambulatory medical device to determine patient status (e.g., a patient response metric) in response to an implemented set of parameter settings, such as a series of different sets of parameter settings received or authorized at a time of implant or initial programming.

For example, the systems and methods can be configured to determine a value or trend indicative of patient status in response to a first set of parameter settings (e.g., determining if the first set of parameter settings are beneficial to the patient or if a change in parameter settings are needed). If the determined patient status to the first set of parameter settings is below a threshold (e.g., an expected patient status or a relative threshold determined as a function of a previous patient status, etc.), a second set of parameter settings can be implemented remotely by the medical device system through the ambulatory medical device. Like with respect to the first set of parameter settings, the systems and methods can be configured to determine patient status in response to updated or changed parameter settings (e.g., the second set of parameter settings, etc.) to determine if the updated or changed parameter settings are beneficial to the patient or if additional changes in parameter settings or additional follow-up is needed. In certain examples, the determined patient status can be used to trigger an alert to a clinician or an adjustment to a follow-up schedule, such as if a value of the determined patient status is above or below one or more thresholds associated with an alert or follow-up requirement, etc.

As therapy progresses, physiologic characteristics of the heart change. Accordingly, even after an initial therapy has been optimized, it is important to periodically reassess and re-optimize therapy and parameter settings at extended intervals (e.g., yearly, etc.). Accordingly, even if patient status is above a healthy threshold, one or more parameter settings or modes can be adjusted to determine positive or negative patient response to the adjustments, such as to guide additional programming changes to continue to provide optimal therapy or parameter settings.

In addition, an automated “operation system test” or “system self-evaluation” on device function or parameter settings can confirm continued optimal operation. In an example, one or more parameter settings can be changed or adjusted and one or more indications of patient condition (e.g., patient response metrics, indications of patient status, risk indications, etc.) can be determined in response thereto. In an example, if the indication of patient condition falls or deteriorates in response to the change or adjustment, device function or parameter settings can be programmed to revert to the previous operation. In other examples, one or more other changes or adjustments can be programmed, in contrast to such changes that provided the previous deterioration in patient condition, and a responsive indication of patient condition can be determined to guide additional changes, adjustments, or reversion to previous operation.

In certain examples, a clinician or a user can implement a stop action to revert or rollback a change in parameter settings in response to negative changes in patient response metrics (e.g., rates, thresholds, impedance, adverse medical events, etc.) after a change. Such stop action can be received by one or more circuits, for example, through an external or a remote device, and can include an open-ended stop action to revert to a previous operation or a time-based stop action allowing a user to provide a stop action to be implemented for a specific post-change time period (e.g., several hours, up to a day, etc.). In other examples, patient response metrics after a change in parameter settings can be evaluated by an external or remote device to recommend or implement a stop action or provide one or more alerts, etc., in response to negative or adverse findings.

Advantages of the systems and methods described herein include, among other things, optimized power usage by the ambulatory medical device, extending a usable lifespan of the limited power resources (e.g., battery) by more quickly optimizing therapy through successive changes in parameter settings, additionally include optimization of clinician resources and follow-up scheduling, as well as reducing the need for patient self-reporting of status following a change in parameter settings, allowing a more responsive and objective reporting and management system.

Implant and follow-up schedules for an ambulatory medical device can vary significantly among patients, devices, or clinicians from time (0) at a time of implant through a first time period (e.g., 1 year) of evaluation and operation. A traditional initial follow-up (in-clinic) can occur at 1 month (e.g., 4 weeks) after implant with subsequent follow-ups (in-clinic) at 6 months and 1 year. Subsequent follow-ups can continue after 1 year at this (e.g., every 6 months) or other durations. Changes to parameter settings can be determined and programmed to the ambulatory medical device by a clinician during follow-up appointments, with additional initial follow-up (e.g., 1 month after a parameter setting change) following each change in parameter settings.

Remote reporting can reduce in-clinic follow-up requirements. For example, a report can be determined, such as by a patient management system, and provided to a clinician at a first time period (e.g., including a recovery period and time to determine a baseline response, such as 1 month, etc.) following implant or a change in programming or parameter settings. In this example, the report can be provided remotely, without an in-clinic follow-up appointment, unless required by a clinician, etc. In response to the report, the clinician can program changes to the parameter settings, such as through the patient management system, to be implemented by the ambulatory medical device without an in-clinic follow-up, or alternatively hold existing settings or schedule an in-clinic follow-up if necessary or desired. After a second time period (e.g., a subsequent recovery period, such as an additional 1 month, etc.) after the change in parameter settings, an additional report can be determined and provided to the clinician. In an example, the report can include a determined patient response metric or patient status or condition based upon received physiologic information for the respective time period, or a comparison of the determined patient response metric for the respective time period in contrast to a determined patient response metric for a previous time period, such as a time before implant, or a time before a prior change, etc. In an example, if no subsequent changes are provided or desired by the clinician, traditional in-clinic follow-up can continue 1 year or one or more other time periods or durations. Also, as noted above, it can be important to re-optimize at longer time periods (e.g., such as at 1 year or greater). In certain examples, it can be beneficial to perform re-optimization in the months or weeks prior to a scheduled in-clinic follow-up, such that the changes are before but close in time to a planned in-clinic visit, reducing subsequent follow-up appointments.

In certain examples, changes can be determined automatically by a patient management system, such as through one or more automated systems configured to analyze parameter settings and generate reprogramming recommendations for ambulatory medical devices to optimize or improve patient condition. In one example, such as with respect to cardiac resynchronization therapy patients, patient condition can be determined with respect to a percentage of confirmed cardiac capture resulting from pacing stimulation, etc. In certain examples, a clinician, at the time of implant or after a recovery period, can authorize a range of settings or a series of settings for the device to cycle and optimize through successive changes and analysis, remotely (e.g., remote from the patient, such as through a cloud-based connection), without in-clinic requirements for each change in parameter settings. If responses are within acceptable guidelines or normal expected values at respective recovery periods (e.g., 1 month after a change, etc.), reports can be placed in the patient medical records without an alert to the clinician until the range or series of settings have been implemented and evaluated, or until a determined patient response exceeds a threshold.

For example, after a recovery period (e.g., 1 month after implant), a first change can include a change from the recovery or monitoring period to the first therapy, or a change from the first therapy to a second therapy. A second change after a second period (e.g., at 2 months) can include a subsequent change different than the first change. A third change (e.g., at 3 months) can include a subsequent change different than the first or second changes, or in certain examples, if one of the previous therapy or sets of parameter settings provided a better patient response metric (higher if a higher value of the patient response metric indicates a positive patient condition, lower if a higher value of the patient response metric indicates a worsening patient condition), the third change can include reverting back to the therapy having the better patient response metric (e.g., from a second therapy to a first therapy, etc.), and away from the therapy or time period indicating a worse patient status.

Although discussed herein as specific time periods, such as pre-determined time periods, etc., in other examples, the rate of change, follow-up, report, recovery, or implementation of initial or subsequent programming or therapy can include one or more other time periods determined by a clinician, determined by patient response metrics or physiologic information of the patient, or one or more other time periods. In an example, a lock-out period can be implemented by one or more devices or circuits or set by a clinician to prohibit device changes in modes or parameter settings in the lock-out period (e.g., one week, two weeks, etc.) prior to a scheduled follow-up appointment, such as to avoid a changing patient condition during subsequent follow-up which may lead to additional unnecessary device changes, enforcing a lock-out period on the device side. In other examples, a follow-up schedule can be modified based on a time of most recent change in parameter settings, such as to avoid a changing patient condition during subsequent follow-up which may lead to additional unnecessary device changes, enforcing the lock-out period on the clinician side.

In an example, the patient management system can be configured to analyze different pacing parameters using artificial intelligence or machine learning, based on received physiologic information or separate therefrom, to identify optimal and suboptimal combinations of pacing parameters corresponding to one or more conditions, such as to optimize patient condition, to improve rates of cardiac capture resulting from pacing stimulation, to eliminate or reduce periods of suboptimal, missed, or reduced pacing, etc. Data can be collected and organized for analysis and identification of patterns. Models can be created based on the identified patterns, validated (e.g., using a percentage of confirmed LV cardiac capture, etc.), stored, and deployed. Additionally, deployed models can be monitored and updated as additional data is collected, including retraining as needed.

Medical device systems can analyze physiologic information between patients or with respect to one or more clinical thresholds to determine patient condition and optimize device settings. In other examples, analysis can focus on differences between the parameter settings themselves (e.g., without respect to patient physiologic information, determined indications of cardiac capture or reduced pacing, patient demographics, patient history, etc.), such that a determined similarity between different parameter settings for the same or different patients can be analyzed to identify sub-optimal settings or combinations of settings that may result in suboptimal, missed, or reduced pacing. In certain examples, parameter settings can be additionally analyzed with respect to one or more of patient physiologic information (e.g., to identify similar patients, etc.), determined indications of cardiac capture or reduced pacing, patient demographics, patient history, or combinations thereof.

Clinicians have broad discretion in determining and implementing parameter settings of medical devices (e.g., CRT devices, etc.) but often follow published literature and guidelines or specific device limits. However, as recommendations change or new therapies, modes, parameters, or settings are introduced, it takes time for such literature or changes in such literature to become widely understood and adopted. For example, certain clinicians may have determined a specific set of parameter settings to optimize pacing in certain patient populations that differ from the previous literature or clinician training. Analysis of settings on a between-patient or between-clinician basis with respect to optimized pacing or capture can identify and determine different combinations of settings and distribute recommended sets of parameter settings more quickly than existing literature. Additionally, whereas clinicians focus on certain parameters, with access to a complete set of parameter settings across large numbers of patients, correlation between seemingly irrelevant parameters in combination with others can be determined that impact cardiac capture rates, improving pacing and cardiac resynchronization therapy, patient outcomes, device performance and efficiency, and communication of leading clinical data more quickly to clinician populations.

In an example, a desired clinical outcome can be provided to the patient management system, such as to optimize cardiac capture. Once the desired clinical outcome is received, changes to therapies or parameter settings can be determined automatically by the patient management system, such as through one or more automated systems configured to analyze parameter settings and generate reprogramming recommendations for ambulatory medical devices to achieve the desired clinical outcome, for example, by optimizing parameter settings to improve a percentage of confirmed cardiac capture resulting from pacing stimulation, etc.

### Artificial Intelligence

Artificial intelligence, particularly machine learning and other techniques, can effectuate the speed and analysis of identifying optimal settings and determining differences between different sets of parameter settings, in combination with physiologic information of the patient (such as determination of patient status, e.g., improving or worsening, etc.) or separate therefrom, taking into account rates of cardiac capture in specific patients or across populations. In addition, separate from tracking rates of cardiac capture for specific patients or patients having specific demographics, disease states, or patient conditions, rates for specific clinicians can be analyzed and determined to identify clinicians having more successful rates of cardiac capture across patients or patient groups.

For example, based on a specific desired output, such as optimizing cardiac resynchronization therapy by effectuating cardiac capture, etc., pacing parameter settings can be analyzed to identify or determine specific parameters or combinations thereof that are more likely correspond to unconfirmed or missed cardiac capture. In an example, although parameter settings often start from a default condition and are separately selectable and adjustable by a clinician, combinations of parameters often ideally move together. In an example, if one parameter is adjusted and a second is not, but adjustment of the second often provides optimal cardiac resynchronization therapy, detection of the second not being adjusted can trigger a recommendation to the clinician to adjust the second parameter. In other examples, such parameters can be adjusted automatically by the system with notice provided to the clinician of such change for review or approval.

In other examples, such as first programming after implant, or situations where patient physiologic information has not been previously recorded or is otherwise unavailable, proposed parameter settings can be recommended based on other information about the patient, such as age, gender, medications, co-morbidities, diagnosed conditions or disease states or progressions, or other information medical history information separate from sensed physiologic information. In this way, the first programmed values for a specific patient can differ from default values for all patients, potentially improving the speed of attaining optimal programming and reducing wasted resources associated with suboptimal operation.

Additionally, optimal parameter settings, or suggested combinations of parameter settings to optimize cardiac resynchronization therapy, may adjust over time, just as the parameters and settings themselves. In certain examples, optimal combinations of parameter settings or suggestions to optimize parameters settings for a particular patient can be provided to a clinician, such as during follow-up or report. In other examples, suboptimal pacing, including unconfirmed or confirmed missed cardiac capture, or a determined patient status or condition lower than a threshold or an expected improvement can trigger analysis and recommendation.

Periods of suboptimal therapy can be harmful to patients but may also lead to inefficient use of device resources including periods of stimulation by the device that may not provide a desired physiologic response, effectively wasting limited device resources. Identifying potentially less effective or ineffective parameter settings or combinations of parameter settings and providing a recommendation of one or more programming changes to improve pacing can result in a more efficient use of device resources while also improving patient therapy. Accordingly, identification of suboptimal settings and generating reprogramming recommendations can improve operation of the underlying hardware.

Parameter settings can be tracked, including patterns of changes across different patients and resulting impact, for example, on patient condition, cardiac capture, etc. Capture can include confirmed capture of one or more chambers, such as confirmed LV cardiac capture, confirmed RV cardiac capture, confirmed RA cardiac capture, or combinations thereof (e.g., confirmed Bi-V cardiac capture, including RV and LV, confirmed LV-only, etc.). In an example, confirmed cardiac capture in less than 98% of cardiac beats (or in other examples, less than 95% or less than 90%) over a period of time, such as a week, a day, a group of successive beats, etc., can trigger an alert or notification and analysis or re-analysis of device parameter settings. In other examples, a reduced trend of confirmed cardiac capture over time, or a sudden loss of cardiac capture below a threshold (e.g., from above 98% to lower than 90%, 80%, 50%, 20%, 10%, or to 0%, etc.) can trigger an alert or notification and analysis or re-analysis of device parameter settings. In other examples, all sets of parameter settings can be analyzed with respect to model parameter settings to identify suboptimal programming and suggest changes, in certain examples, additionally with respect to confirmed cardiac capture percentage. For example, if key opinion leaders change a model set of parameter settings, even in situations where a patient has confirmed cardiac capture at 98% or above, a notification can be provided to a clinician illustrating the differences and impact of such to the medical device and the patient.

In an example, the one or more pre-trained machine learning models can be trained, in certain examples, such as described herein. For example, the determined patient response metric and parameter settings can be input into one or more pre-trained machine learning models trained to compare the received parameter settings to stored model parameter settings from one or more other implantable medical devices corresponding to one or more other patients and to identify one or more differences between the parameter settings of the implantable medical device and the stored model parameter settings of the one or more other implantable medical devices and different determined patient response metrics. Upon obtaining an output from the one or more pre-trained machine learning models indicating the identified one or more differences between the parameter settings of the implantable medical device and parameter settings of the one or more other implantable medical devices, a programming recommendation can be generated for the implantable medical device to improve patient condition (e.g., cardiac capture) for the patient based on the identified one or more differences, or device performance (e.g., resource usage) without negatively impacting patient condition, etc.

In other examples, if the determined patient response metric indicates that the existing therapy is stable or changing, etc., one or more modes or functions of the implantable or ambulatory medical device can be altered to increase or decrease a power consumption or sensing or storage capability of the implantable or ambulatory medical device. For example, one or more hardware limitations can be adjusted, such as to, among others: sense or receive more or less physiologic information of the patient; increase or decrease communication frequency between the implantable or ambulatory medical device and an external device (e.g., remote device, programmer, etc.), such as to increase or reduce the frequency of patient monitoring, etc.; switch to a different power or resource intensive monitoring algorithm; etc. In an example, if the determined patient response metric is stable after an extended period of time (e.g., greater than 6 months, greater than one year, greater than two years, five years, etc.), programming recommendations can be provided to a clinician for review, or implemented automatically, to reduce power consumption or extend device lifespan without negatively impacting the patient. In other examples, if the determined patient response metric has been stable for an extended period of time but then starts to drift (e.g., starts to slowly decrease, but not yet triggering any alert thresholds, etc.), one or more modes or parameter settings can be adjusted to determine if different parameter settings can reduce drift or improve patient condition before a subsequent follow-up. In certain examples, changes can be prohibited if the determined patient response metric is not stable at a time of a proposed change.

In addition, during programming or reprogramming of medical devices, related parameter settings can be identified and grouped, such as to suggest corresponding changes (e.g., during initial programming, during follow-up sessions, etc.), where one parameter setting is changed, suggesting corresponding changes to one or more other programmer settings to optimize therapy to the patient. In addition, values of such parameter settings can be analyzed to suggest not only specific parameter settings to program or reprogram, but values of different parameters based upon proposed changes. For example, if changing one parameter by a first amount, a recommendation can be provided to change a second parameter by a second amount. In contrast, if changing the one parameter by an amount greater than the first amount, the recommendation can be changed to change the second or one or more other parameters by an among different than the second amount, greater or less, depending on the specific parameters.

A recommendation system, such as comprising one or more assessment circuits, can utilize artificial intelligence and machine learning models to identify corresponding changes, for example, corresponding to positive outcomes (or away from negative outcomes), such as with respect to patient condition, confirmed cardiac capture percentages of cardiac beats, or one or more other desired clinical outcomes in other patients. In certain examples, parameter settings by key opinion leaders can be weighed more heavily than parameter settings by other clinicians. In other examples, specific models based on parameter settings of key opinion leaders, including individual clinicians or groups of clinicians at the forefront of thought leadership in the field of cardiac rhythm management therapy, can be suggested, separate from other clinicians. Combinations of parameter settings (e.g., including patterns, values, etc.) can be identified and validated to optimize patient condition, cardiac capture, etc., and validated identified parameter settings can be recommended as optimal values to clinicians, such as when programming or reprogramming medical devices, including implantable medical devices, etc. In certain examples, validation can include combinations of human and machine validation confirmed based on stored information and recorded patient outcomes.

In other examples, other potential suboptimal pacing scenarios can be improved using the systems and methods described herein, such as identifying suboptimal parameter settings associated with high pacing (e.g., a percentage of paced beats above a high or desired pacing threshold) in an implantable cardioverter defibrillator (ICD) (e.g., not a cardiac rhythm management device, but a defibrillator), or chronic overpacing in one or more ambulatory medical devices, where a patient condition could have changed such that existing settings, although resulting in cardiac capture, are not required any longer. For example, patient physiologic information, such as heart sound information (e.g., S1 amplitude, providing an indication of cardiac contractility of a ventricle, etc.) can be used to triage patients and determine whether periods of intrinsic activity should be allowed, or if one or more parameter settings should be adjusted or recommended commensurate with a change in the patient physiologic information or patient status determined using such information.

### Feature Extraction

FIG. 1 illustrates an example 100 of a table 101 of parameter settings and a reduction 102 of different combinations of parameter settings. In an example, specific parameter settings for a medical device can be represented by changes or deviation from a mode (e.g., a most frequent, or in other examples another statistical parameter, such as an average, a median, etc.) for a respective parameter setting from other medical devices, other medical devices in similar patients (e.g., grouped by disease, physiologic information, patient status, or combinations thereof), or the same medical device in a patient but at a different times. For example, whereas each medical device has a set of parameter settings, respective medical devices also have different sets of parameter settings at different times.

A kernel is a type of function used in various AI and machine learning algorithms that enables an algorithm to operate in high-dimension space without computing the coordinates of the data in that space. For example, a kernel can compute a dot product of multiple vectors without having to compute the coordinates of the points in that space, saving computational resources. In an example, a kernel can be used to extract and store patterns in different sets of parameter settings of cardiac resynchronization therapy or other medical devices (e.g., an implantable cardioverter defibrillator (ICD), etc.) and to identify rules for different sets of parameters for specific sets or classes of devices performing similar functions, in certain examples, further tailored to specific patients based on physiologic information of such patients.

In an example, parameter settings for one medical device are illustrated in the table 101 for example, across different office visits or sets of programming changes, etc. The changes in settings are illustrated with a 1 or a 0 in the table 101. For example, going from a 0 to a 1 can indicate that a previous setting was not utilized in the 0 state but is not utilized in the 1 state. In other examples, 0 and 1 can include a range of values that the parameter is outside of or a value that the parameter is above or below, etc., such as indicated with a 0, or a range of values that the parameter is now inside of or a value that the parameter is below or above, etc., such as indicated with a 1.

Columns or rows of the table 101, or in certain examples the table 101 itself, depending on organization, can be treated as vectors. In an example, columns A-D illustrate four different table entries. However, the four table entries A-X illustrate a number of parameter settings, P1-PN, with P1 having two states (P1A and P1B), reflecting different changes, for example, from a mode (most used value or setting for a particular parameter across a group of patients, such as in the training data, etc.) to a first value, illustrated as P1A, or from the mode to a second value, illustrated as P1B.

In a particular example, P1 can include an offset value, such as a sensed atrioventricular delay (AVD) offset value of 30, 40, or 50 ms, with a mode of 30 ms. P1A can represent a change from 30 ms to 40 ms, and P1B can represent a change from 30 ms to 50 ms. In other examples, additional table entries can be included, such as P1C illustrating a change from 40 ms to 50 ms, or other values, parameters, etc. P2 through PN can include other parameter settings, such as particular sensing or tracking modes with 0 representing the mode (e.g., on or off) and 1 representing a change from the mode (e.g., off or on, respectively). As with P1 including P1A and P1B, P2, . . . PN can also include other states as needed.

Parameter settings can be transformed into a table format or vectors, such as described herein (e.g., variance from a mode), and different combinations of settings and changes resulting therefrom (e.g., % of atrial or ventricular cardiac capture, etc.) can be evaluated to identify sets of parameter values or corresponding changes that represent the greatest positive impact, or positive impact above a threshold.

For example, changes in parameter settings can be evaluated to identify candidate rules or parameters values or combinations of parameter values that exceed a minimum support value (a) above a threshold (e.g., an upper percentile of response, etc.) to produce a reduction 102 of combinations of parameter settings. The minimum support value can be a function of frequency, impact, or combinations thereof.

In certain examples, upward closed analysis (or other analysis) can be used to reduce the overall work required to identify combinations of parameter values and associated rules. Upward closed analysis generally refers to a property where, if first and second parameter values are below a minimum support value (e.g., P(A)<α and P(B)<α), then any combination of parameters including the first and second parameter values will be below the minimum support value (P(A,B)<α). Based on such analysis, the reduction 102 illustrates that if P(X)<α, then all combinations including P(X) can be excluded from analysis. Similarly, if P(B,C)<α, then all combinations including P(B,C) can be excluded from analysis.

Although described herein with respect to upward closed analysis, in other examples other types of analysis can similarly be used, such as lower closed analysis or other order or lattice theory elements or properties, etc. Additionally, although discussed herein with respect to a single patient or a single medical device, in other examples, parameters can be evaluated across different patients, across different medical devices, or combinations thereof.

### Machine Learning Model

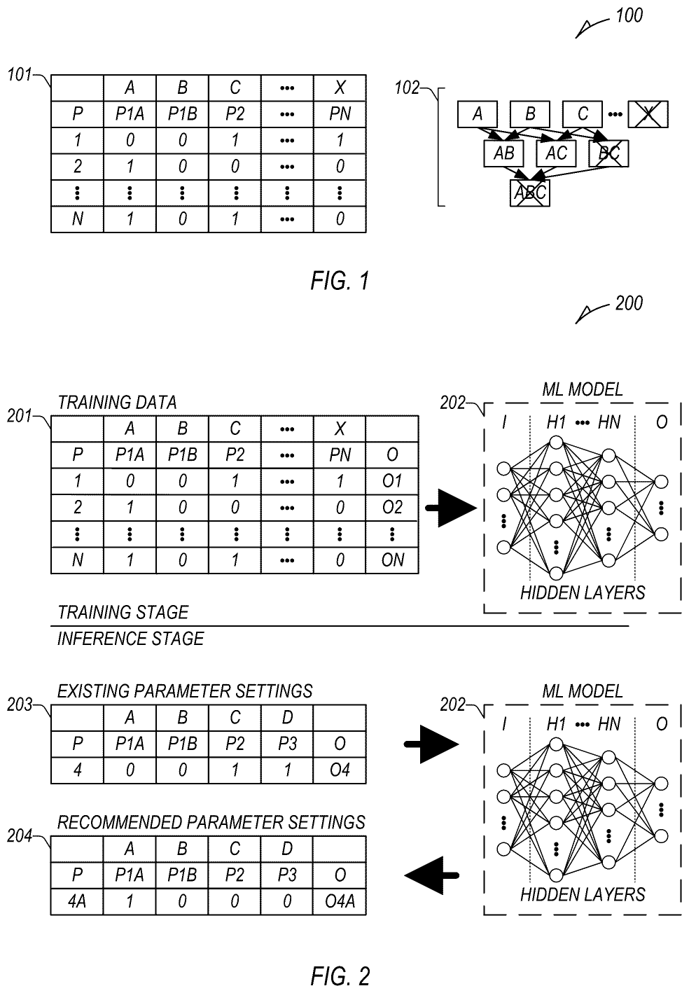

FIG. 2 illustrates a diagram 200 of an example machine learning model 202 trained to receive, as input, training data 201, such as parameter settings from one or more ambulatory medical devices, with respect to one or more patients, and in certain examples physiologic information associated with respective parameter settings, such as patient condition of the one or more patients, associated cardiac capture rates of different patients, or other physiologic information, etc., to identify optimal or suboptimal parameter settings or combinations of parameter settings corresponding to a desired clinical outcome, such as successful cardiac resynchronization therapy, patient status or condition, etc., and to generate an output indicating proposed parameter settings to optimize the desired clinical outcome (e.g., to optimize cardiac resynchronization therapy, such as to reduce suboptimal, missed, or reduced pacing based on existing parameter settings, to improve patient condition, etc.).

In the training (or learning) stage of FIG. 2, the training data 201 can be applied to a machine learning algorithm to train the machine learning model 202. In an example, the machine learning model 202 can include (1) a digital model of a patient, such as based on received physiologic information of a patient, (2) a digital model of an ambulatory medical device, such as based on one or more parameter settings or combinations of parameter settings, configuration, or type of the ambulatory medical device or a plurality of other ambulatory medical devices or in certain examples based on received physiologic information from one or a plurality of patients, or (3) combinations or permutations thereof.

The training data 201 can include parameter settings (e.g., P1A, P1B, P2, . . . PN, etc., such as described in FIG. 2) from a number of different medical devices (e.g., 1-N, etc.). Although illustrated as parameters settings (e.g., P1A, P1B, P2, . . . PN) and medical devices (1-N), in practice the training data 201 can include substantially more parameter settings and medical devices, such as tens or hundreds of parameter settings and hundreds or thousands of medical devices. In certain examples, the same medical device can provide parameter settings at different times. In certain examples, each vector or set of parameter settings can include an outcome (O) or assessment information, such as patient physiologic information (e.g., cardiac capture information, etc.) or other information of or about a patient associated with the respective parameter settings. In certain examples, the assessment can be provided by or validated by a user, such as based on direct measurement of patient physiologic information, etc.

The machine learning model 202 can be trained using supervised learning, unsupervised learning, or reinforcement learning. Examples of machine learning model architectures and algorithms may include, for example, decision trees, neural networks, support vector machines, or deep neural networks, etc. Examples of deep neural networks can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the machine learning model may be performed continuously or periodically, or in near real time as additional patient data are made available. The training involves algorithmically adjusting one or more model parameters or parameter settings, until the model being trained satisfies a specified training convergence criterion. The machine learning model 202 can establish a correspondence between parameter settings or combinations of parameter settings and patient condition, cardiac capture, etc.

For example, with respect to an outcome, such as cardiac capture, a machine learning model can be trained to analyze physiological signal data and detect a respective outcome, such as cardiac capture or successful or optimal cardiac resynchronization therapy, or correspondingly, suboptimal cardiac resynchronization therapy. To train such a model, for example with respect to cardiac capture, a dataset is assembled containing sample patient physiologic information annotated by medical experts to identify successful cardiac capture and correspondingly, unconfirmed cardiac capture or unsuccessful cardiac capture. This annotated training dataset is then used to train the machine learning model 202 using a supervised learning approach, such as by algorithmically adjusting internal parameters to map the input patient physiologic information to the expert-applied labels. Various machine learning algorithms can be used, such as described above. The training process continues until the model achieves a high accuracy in classifying optimal or sub-optimal cardiac resynchronization therapy.

The training and utilization of machine learning models to determine indications of patient condition, cardiac capture, etc., and to identify parameters or combinations of parameters associated with the desired clinical outcome, such as optimal or sub-optimal cardiac resynchronization therapy, etc., can depend on the specific type of ambulatory medical device being used and the nature of the physiological signal data it collects. Once trained, the machine learning model can receive new parameter settings and patient physiologic information as input and automatically detect if the patterns reflect the desired clinical outcome, such as optimal or sub-optimal cardiac resynchronization therapy, etc., and to identify specific parameter settings or combinations of parameter settings to change to achieve the desired clinical outcome. This allows advanced analysis without needing to hard-code detection criteria. The model can be periodically retrained on new data to optimize parameter settings over time.

In the inference stage of FIG. 2, for example, after the machine learning model 202 has been trained to reliably analyze parameter settings, patient physiologic information, or combinations thereof, and to identify and suggest programming or reprogramming recommendations to optimize a desired outcome or patient condition, such as cardiac resynchronization therapy, etc., the machine learning model 202 can be deployed for use in a production setting. Accordingly, existing parameter settings 203 (P1A, P1B, P2, . . . PN) of an ambulatory medical device (4) can be provided as input to the machine learning model 202, such as at a remote server computer (e.g., a cloud-based server or processor, etc.), in certain examples including patient physiologic information (O4). The machine learning model 202 can generate one or more outputs, including recommended parameter settings 204, indicating reprogramming recommendations for the ambulatory medical device (4A) and in certain examples an indication of outcome expected by such reprogramming recommendations (O4A).

In certain examples, the received patient physiologic information can be used as input to a rule-based recommendation engine for generating a recommendation to program or reprogram an ambulatory medical device, for example, when one or more parameter settings or combinations of parameter settings are identified by the machine learning analysis as suboptimal or having a high likelihood of resulting in suboptimal cardiac resynchronization therapy. In certain examples, a number of different parameter settings can be simulated to predict respective outcomes and the different parameter settings or combinations of parameter settings can be prioritized by the predicted respective outcomes.

In other examples, a desired outcome can be used as input to the rule-based recommendation engine for generating a recommendation to program or reprogram the ambulatory medical device. Different parameter settings can be simulated, in certain examples on different ambulatory medical devices, to predict respective outcomes. The predicted respective outcomes can be compared to the input desired outcome, and different parameter settings or combinations of parameter settings or different types of ambulatory medical devices, configurations, or therapies can be recommended based on a comparison of the predicted respective outcomes to the input desired outcome.

### Programming Recommendations

Consistent with some embodiments, a programming or reprogramming recommendation may involve a recommendation to modify or reprogram the device to use one or more different settings in sensing events, activity, or physiologic information, such as one or more blanking periods, thresholds, etc., or in providing stimulation, such as one or more intervals, delays, stimulation amplitudes, selected electrodes or vectors, etc. In certain examples, the recommendation to modify or reprogram the device can include a recommendation to use one or more different sensitivity settings or modes different from the current sensitivity setting or mode. With some embodiments, each sensitivity setting, or sensitivity mode is associated with one or more predefined threshold values. Accordingly, when a device is programmed or reprogrammed to operate in a new sensitivity mode, one or more of the predefined threshold values can change, thereby increasing or decreasing the sensitivity for detecting a particular event or activity. The programming or reprogramming recommendation may be presented via a user interface of a software application to a clinician, who may undertake the task of reprogramming the device for a patient. The clinician can then evaluate the recommendation and program or reprogram the ambulatory medical device accordingly. This allows the clinician to validate any proposed changes to the device based on their expert judgment. In other examples, the programming or reprogramming recommendation can be automatically applied within limits, such as previously validated by the clinician, etc., or directed based on physiologic response of the patient, such as determined by one or more patient response metrics indicative of patient response to different cardiac rhythm management therapy.

Embodiments of the present invention provide numerous technical advantages for optimizing programming of and therapy delivery by ambulatory medical devices. By periodically evaluating collected physiological signal data using advanced machine learning models, embodiments of the invention enable closed-loop optimization of operation of the ambulatory medical device over time. This allows enhancing operation of the ambulatory medical device compared to relying solely on static detection settings programmed at implantation. The machine learning analysis can identify suboptimal parameter settings missed by the ambulatory medical device or the clinician programming the ambulatory medical device and recommend adjustments to improve operation when appropriate. The system can adapt parameter settings to the individual patient commensurate with patient status or therapy efficacy, providing more accurate therapy and without requiring constant manual reprogramming, reducing workload for clinicians while optimizing device performance and increasing the speed of training and sharing updated protocols and guidance.

### Parameter Settings

Parameter settings differ based on the type of medical devices and in certain examples can include different modes of therapy. For example, different modes of cardiac resynchronization pacing include DDD and DDDR pacing, among others. The first “D” in DDD pacing represents dual (D) chamber (atrium and ventricle) pacing, the second “D” represents dual (D) chamber sensing, and the third “D” represents dual (D) chamber response to sensing in coordinating contraction of the heart and improve cardiac function. The additional “R” in DDDR pacing represents rate (R) modulation, where the medical device can adjust the pacing rate based on physiologic information indicating activity or need (e.g., activity, breathing rate, etc.).

Parameter settings can include, among others: Sensed Atrioventricular Delay Offset (SenAVDIyOffset or SAVDO), which adjusts the delay after a sensed atrial event; Atrioventricular Dynamic Minimum (AVDynMin or AVDM), which sets the minimum dynamic atrioventricular delay; Atrioventricular Delay Fixed (AVDlyFix or AVDF), which is a fixed atrioventricular delay setting; Atrioventricular Dynamic Maximum (AVDynMax or AVDM), which defines the maximum dynamic atrioventricular delay; Lower Rate Interval (LRLIntvl or LRLI), which sets the minimum pacing rate for the device; Left Ventricular Offset (LVOffset or LVO), which adjusts the timing of left ventricular pacing in relation to right ventricular pacing; Atrioventricular Dynamic Enable (AVDynEnbl or AVDE), which enables dynamic adjustment of the AVD; and Maximum Tracking Rate Interval (MTRIntvl or MTRI), the fastest interval at which the device will track atrial rates.

Additional parameter settings can include: Atrial Tachy Response Mode (ATRMode or ATRM), which defines the operational mode of the atrial channel; Biventricular Trigger Enable (BiVTrigEnbl or BVTE), which activates the biventricular pacing trigger; Ventricular Tachycardia Zone Rate (VTZoneRate or VTZR), which sets the rate threshold for detecting ventricular tachycardia; Atrial Tachy Response Trigger Rate (ATRTrigRt or ATRTR), which is the rate at which Atrial Tachy Response Mode is triggered; Maximum Sensor Rate Interval (MSRIntvl or MSRI), the maximum rate at which the device will pace in response to sensor input; Ventricular Tachycardia 1 Zone Rate (VT1ZoneRate or VT1ZR), which specifies the rate for a particular zone of ventricular tachycardia detection; Number of Ventricular Zones (NumVZones or NVZ), which determines how many zones are used for ventricular tachyarrhythmia detection; Ventricular Fibrillation Zone Rate (VFZoneRate or VFZR), which sets the rate threshold for detecting ventricular fibrillation; Atrial Tachy Response Ventricular Rate Regulation Response (ATRVRRResp or ATRVRRR), a setting that adjusts the ventricular pacing rate in response to atrial rate; Atrial Tachy Response Biventricular Trigger Enable (ATRBiVTrigEnbl or ATRBVTE), which allows for biventricular pacing in response to atrial rate; Atrial Tachy Response Lower Rate Limit (ATRLRL), which sets the minimum pacing rate in ATR mode; Tachycardia Mode (TachyMode or TM), which defines the operational mode for tachycardia detection and therapy; Respiration Rate Trend Enable (RRTenable or RRT), which activates the respiration rate tracking feature; Atrial Tachy Response Pacing Chamber (ATRPaceCham or ATRPC), which specifies the chamber to be paced in atrial tachy mode; and Sensing Mode (SenseMode), which determines how the device senses cardiac events.

### Related Parameter Settings

The different parameter settings described above can have different having different relationships with respect to each other. For example, based on analysis of multiple patients using feature extraction, such as illustrated and described in FIG. 1, and machine learning, such as described in FIG. 2, different combinations or subcombinations can be determined having closer relationships than others, such that if one parameter is going to be adjusted, the patient is likely to benefit from adjustment of a second or one or more other parameters. Different subgroups are illustrated below, the first having a closer relationship than the second, the second closer than the third, etc.

A first subgroup can include the following parameters, listed in order of relationship (e.g., parameters (1) and (2) are closer than (1) and (9), etc.), in certain examples enforced using one or more rules executed by the programmer: (1) Sensing Mode (SenseMode) and Atrial Tachy Response Pacing Chamber (ATRPaceCham or ATRPC) should be adjusted or recommended to be adjusted together, for example, on the same or sequential programming screens of the programmer, followed closely by; (2) Respiration Rate Trend Enable (RRTenable or RRT); (3) Tachycardia Mode (TachyMode or TM), (4) Atrial Tachy Response Lower Rate Limit (ATRLRL). In certain examples, the first subgroup can additionally include the following parameters: (5) Atrial Tachy Response Ventricular Rate Regulation Response (ATRVRRResp or ATRVRRR) and (6) Atrial Tachy Response Biventricular Trigger Enable (ATRBiVTrigEnbl or ATRBVTE). Somewhat unexpectedly, (7) Number of Ventricular Zones (NumVZones or NVZ); (8) Ventricular Fibrillation Zone Rate (VFZoneRate or VFZR); and (9) Ventricular Tachycardia 1 Zone Rate (VT1ZoneRate or VT1ZR) are more closely related to each other than (1)-(6).

A second subgroup can include the first subgroup (e.g., parameters (1)-(9)) and additionally the following: (10) Maximum Sensor Rate Interval (MSRIntvl or MSRI); (11) Atrial Tachy Response Trigger Rate (ATRTrigRt or ATRTR); (12) Ventricular Tachycardia Zone Rate (VTZoneRate or VTZR); (13) Biventricular Trigger Enable (BiVTrigEnbl or BVTE); and (14) Atrial Tachy Response Mode (ATRMode or ATRM).

A third subgroup can include the following parameters: Atrioventricular Delay Fixed (AVDlyFix or AVDF) and Atrioventricular Dynamic Maximum (AVDynMax or AVDM), suggesting that such parameter settings should be grouped using one or more rules. A fourth subgroup can include the third subgroup and additionally the following parameters: Sensed Atrioventricular Delay Offset (SenAVDIyOffset or SAVDO) and Atrioventricular Dynamic Minimum (AVDynMin or AVDM). Each of the identified subgroups have significant identified relationships and can be changed or alerted to be changed together, such that if one setting from the group is adjusted, the others are alerted for consideration or adjustment.

As described herein, patterns or combinations of parameter settings can be identified and validated as affecting or impacting cardiac resynchronization therapy. For example, groups of parameters can be identified having a higher percentage of cardiac capture rates than other parameters, or groups that, once such parameter settings are implemented, show an increase in rates of cardiac capture. Once identified, individual values for specific parameter settings can be toggled or changed and the impact to cardiac resynchronization therapy across a population or number of patients can be analyzed to determine positive or negative impact on delivered therapy, such as evidenced by rates of cardiac capture or other patient physiologic information.

In one example, a first group of parameter settings was identified as: VTZoneRate=300 ms, SenAVDlyOffset=40 ms, NumVZones=3. Once implemented, an increase in LV cardiac capture was detected. To validate the identified group, cardiac capture rates (e.g., LV pacing % (successful capture) and RV pacing %) are analyzed by toggling values of the parameter settings to or away from default or mode values for each setting and comparing cardiac capture rates for different values. If the cardiac capture rates do not significantly change (e.g., increase or decrease less than a threshold percentage) with respect to the toggle, the specific parameter value can be identified as not having a substantial impact. However, if the cardiac capture rates change (e.g., an increase or decrease more than a threshold percentage) with respect to the toggle, the specific parameter value can be identified as having a substantial impact. In this example, the VTZoneRate=300 ms parameter (e.g., toggled with respect to an example mode value of 400 ms, etc.) provided a more substantial positive impact on LV cardiac capture rate than the other parameters, though less than the combination of the first group of parameter settings in aggregate, validating the combination.

In contrast, in another example, a second group of parameter settings was identified as: BiVTrigEnbl=1, SenAVDlyOffset=50 ms, VFZoneRate=273 ms, NumVZones=3. Once implemented, an increase in RV cardiac capture was detected. To validate the identified group, cardiac capture rates are analyzed by toggling values of the parameter settings to or away from default or mode values for each setting and comparing cardiac capture rates for different values. In this example, the BiVTrigEnbl=1 parameter (e.g., toggled from 1 to 0) provided a substantial positive impact on LV cardiac capture and the NumVZones=3 parameter (e.g., toggled from 3 to 2, etc.) provided a substantial positive impact on RV cardiac capture, though each less than the combination of the second group of parameter settings in aggregate, validating the combination.

CRT Vs. MSP Mode Switch

Certain heart failure patients respond to (e.g., benefit from) multi-site pacing therapy that do not respond to traditional (non-multi-site pacing) cardiac resynchronization therapy. Other patients do not respond to either. Pacing therapies can be evaluated to ensure that the applied pacing therapy is providing some benefit to the patient, to determine whether or not the pacing therapy should be adjusted, or to determine if the pacing therapy should be transitioned. For example, one study found that 25% to 40% of heart failure patients with myocardial dysfunction did not respond to conventional cardiac resynchronization therapy (e.g., bi-ventricular pacing therapy) by evaluation at 6 months post implant, but also found that over half (51.3%) of non-responders to cardiac resynchronization therapy at 6 months did subsequently respond to 6 months of multi-site pacing therapy in the left ventricle by evaluation at 12 months post implant.

Although the transition from CRT to multi-site pacing therapy in the left ventricle was substantially complication free physiologically, the switch to multi-site pacing therapy from conventional CRT can provide at least some cardiac stress, but also requires additional resources from the implantable medical device. In certain estimates, implementing multi-site pacing therapy in a device capable of multi-site pacing therapy and CRT reduces the estimated life of the device by 11-13%. Even a single six-month evaluation of multi-site pacing therapy on an IMD can impact the usable life of the IMD by a relatively substantial and often unnecessary amount.

In certain examples, physiologic information sensed from the patient in one or more time periods can be used to determine one or more multi-site pacing response metrics, and that such one or more determined multi-site pacing response metrics can be used to provide an alert or notification or otherwise control transition between different medical device modes (e.g., a first stimulation mode, a second stimulation mode, etc.), including, in certain examples, to: (1) control transition of a non-multi-site pacing therapy mode to a multi-site pacing therapy mode; (2) control transition of the multi-site pacing therapy mode to the non-multi-site pacing therapy mode; or (3) enable or disable the multi-site pacing therapy mode.

Moreover, physiologic information sensed from the patient subsequent to implementing a stimulation mode can be used to determine a multi-site pacing response metric configured to determine an indication of a predicted patient response to the stimulation mode, such that, in certain examples, a stimulation mode can be evaluated without entering the stimulation mode. For example, specific physiologic information or combinations of physiologic information can be determined to evaluate a stimulation mode or to predict a positive patient response to a particular stimulation mode, such as using a response to another stimulation mode, etc. In an example, physiologic information sensed or detected during a CRT mode can be used to determine if a patient is likely to respond to (e.g., benefit from) a multi-site pacing therapy mode before the multi-site pacing therapy mode is implemented or enabled.

In an example, impedance information (e.g., ITTI), respiration information (e.g., RSBI), or a combination thereof over one or more time periods can be used to determine an indication that a patient will respond to a multi-site pacing therapy mode. A first patient response metric, p1, (e.g., a multi-site pacing response metric) can be determined as follows:

$$( α \times ITTI ) + ( β \times RSBI ) \qquad ( 1 )$$

In equation (1), ITTI is a measure of patient intrathoracic impedance, RSBI is a measure of patient respiratory rate (RR) or frequency to a measure of tidal volume (TV) (e.g., RR/TV, etc.), and α and β are variables. In other examples, the patient response metric can be determined using only one of ITTI or RSBI information, or as different functions of one or more of such physiologic parameters in combination with one or more other physiologic parameters.

In other examples, other patient response metrics can be determined using other physiologic information, such as heart sound information (e.g., S3/S1, etc.), RSBI information, ITTI information, etc. For example, S3/S1, is particularly well correlated to determining if a patient is likely to respond to a CRT mode (or CRT mode or MSP therapy mode), in contrast to being a non-responder (e.g., there is a difference in heart sound information between responders and non-responders). RSBI information is similarly well correlated. A second patient response metric, p2, (e.g., a CRT pacing response metric) can be determined as follows:

$$( α \times ( S 3 / S 1 ) ) + ( β \times RSBI ) \qquad ( 2 )$$

In equation (2), S3 is a third heart sound parameter (e.g., an amplitude or energy of the third heart sound, etc.), S1 is a first heart sound parameter (e.g., an amplitude or energy of the first heart sound, etc.), RSBI is a measure of patient respiratory rate (RR) or frequency to a measure of tidal volume (e.g., RR/TV, etc.), and α and β are variables. In other examples, the patient response metric can be determined using a combination of this or other heart sound information (e.g., other than S3/S1, etc.), using only one of heart sound or RSBI information, using information over different time periods, or as a combination with one or more other physiologic parameters.

In other examples, one or more other patient response metrics can be determined using sensed or received physiologic information of the patient, or combinations of sensed or received physiologic information of the patient, such as one or more of night heart rate information, activity information, determination of patient rest (e.g., a lack of activity, etc.), a multi-sensor HeartLogic™ index, a slope of a minute ventilation (MV) signal, determined AVD parameters, pacing thresholds in or in response to the different therapies or modes, continuous ECG measurements, impedance measurements, occurrence or detection of adverse events (e.g., arrhythmia, etc.), etc. Different modes or parameter settings can be implemented for a time period (e.g., 1 week, 1 month, etc.), the patient response metric can be determined in response thereto, and the therapy can be adjusted accordingly, with reporting and alerts coincident to changes in determined patient response metrics, with changes in parameter settings, or in response to changes exceeding one or more thresholds. In certain examples, patient response metrics can include a comparison of a determined patient response metric pre and post change and can trigger further adjustment or a report to a clinician for review.

### Method Examples

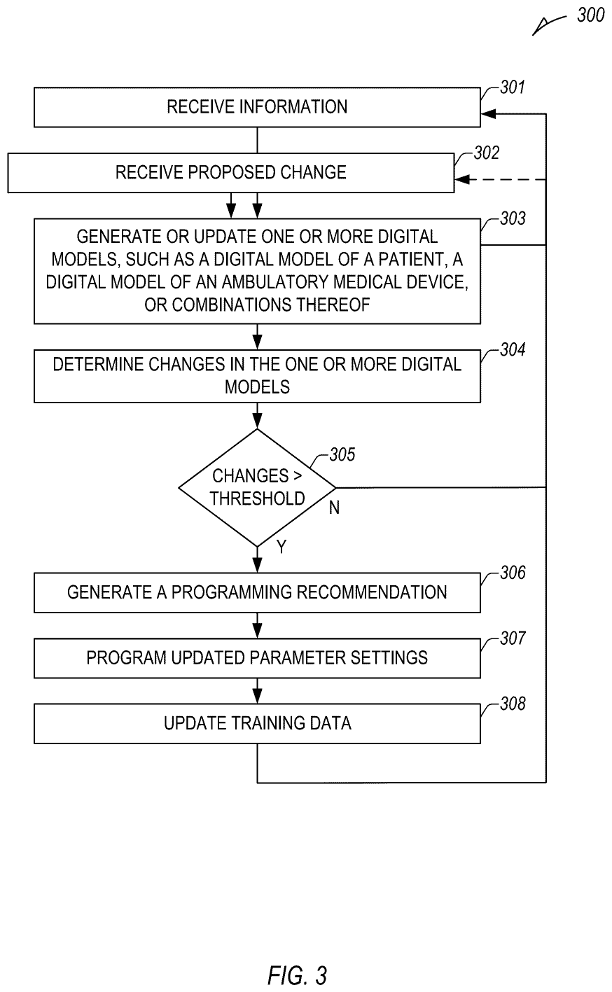

FIG. 3 illustrates an example method 300 for optimizing resources of an ambulatory medical device, such as for evaluating patient condition and programming (e.g., remotely, such as through one or more communication circuits, etc.) an ambulatory medical device to optimize resources of the ambulatory medical device or for predicting an outcome of programming changes to an ambulatory medical device and programming (e.g., remotely) the ambulatory medical device to optimize resources of the ambulatory medical device. The method 300 can be implemented using one or more processors and one or more memory devices storing instructions or using one or more circuits or components as described herein.

At step 301, information can be received, such as using a signal receiver circuit or one or more other components. The information can include one or more parameter settings of an ambulatory medical device (e.g., existing parameter settings, etc.), physiologic information of a patient from the ambulatory medical device, or combinations thereof.

The parameter settings can include settings indicative of a state or mode of the ambulatory medical device, such as parameter settings from a clinician during a programming or reprogramming session, existing parameter settings of an ambulatory medical device, or other parameter settings of or proposed for the ambulatory medical device. For example, parameter settings in a first time period can be representative of a first cardiac rhythm management therapy, implemented at a time of implant (e.g., including a post-implant time period after a received indication of a time of implant of an ambulatory medical device, etc.) or later, such as after a recovery period or after a monitoring period after one or more previous reprogramming instructions. Parameter settings can subsequently be received in a second time period or one or more other time periods subsequent to the first time period, each representative of a specific therapy having defining characteristics, such as a mode or one or more parameter settings. and can be received from an ambulatory medical device or a remote or external device configured to program the ambulatory medical device.

The physiologic information can include physiologic information of the patient, such as one or more types of physiologic information or parameters sensed using one or sensors of the ambulatory medical device associated with the patient, etc. In certain examples, the physiologic information can include information from a specific time period, such as a first time period (e.g., a post-implant time period) or one or more other time periods, such as a second time period, etc., separate from or different than the first time period.

In other examples, the received information can include one or more digital models, such as a digital model of the patient, a digital model of the ambulatory medical device, or combinations thereof, for example, to be updated based on other received information, such as parameter settings of the ambulatory medical device, physiologic information of the patient, etc.

At step 302, a proposed change can be received, such as by the signal receiver circuit or one or more other components. The proposed change can include a proposed programming change to an ambulatory medical device, such as a proposed change to one or more parameter settings, configuration, proposed therapy, etc. In other examples, the proposed change can include a proposed implementation of a programming mode, therapy, or ambulatory medical device not yet associated with or implemented or implanted in the patient.

At step 303, the one or more digital models, such as the digital model of the patient, the digital model of the ambulatory medical device, or combinations thereof can be generated or updated (or in certain examples accessed), such as using an assessment circuit or one or more other components, for example, based on at least a portion of the information received at one or both of step 301 or step 302 or other information derived from the information received at one or both of step 301 or step 302, such as determinations of patient condition or different indications, detected events, information received about the patient, such as demographic information, etc., information from medical records, previous determinations or indications, etc. The digital model of the patient, the digital model of the ambulatory medical device, or combinations thereof can be generated or updated (e.g., continuously) based on information received about the patient, information or parameter settings of the ambulatory medical device, information about one or more other patients, information about one or more other ambulatory medical devices, or combinations thereof.

At step 304, changes in the one or more digital models can be determined, such as using an assessment circuit or one or more other components. For example, at step 303, if one or more digital models are first generated, such one or more models can be updated (e.g., continuously) as additional information is received, such as physiologic information of the patient or one or more other patients, information from or about the ambulatory medical device, the patient or one or more other patients, or one or more other ambulatory medical devices, etc. In certain examples, digital models can be generated or updated using information from specific time periods, such as a first time period and a second subsequent time period, updating the digital models based on a determined difference between the information received from the different time periods.

In other examples, one or more digital models, such as received digital models, can be updated at step 303 using additional received information, such as the physiologic information of the patient or one or more other patients, information from or about the ambulatory medical device, the patient or one or more other patients, or one or more other ambulatory medical devices, etc. As the one or more digital models are updated, changes in the one or more digital models can be determined in certain examples indicating changes in the digital model of the patient, changes in the digital model of the ambulatory medical device, or combinations thereof.

In an example, the determined changes can include or be indicative of a patient response metric determined using changes in the received physiologic information or changes in received information about the ambulatory medical device, such as a function of one or more types or values of physiologic information of the patient or changes in determined parameters of the ambulatory medical device (e.g., estimated remaining lifespan, successful communication, etc.), or combinations or permutations thereof. The patient response metric can be indicative of a patient status for the respective therapy, mode, or parameter settings of a respective time period from which the patient response metric is based.

In an example, the changes can be determined by simulating application of the proposed programming change on the digital model of the patient using the digital model of the ambulatory medical device and predicting a change in the updated digital model of the patient of the proposed programming change based on training data from a plurality of other patients or ambulatory medical devices. In other examples, the changes can be determined by simulating application of the proposed programming change on the digital model of the ambulatory medical device to predict an outcome of the proposed programming change based on training data from a plurality of other ambulatory medical devices. In certain examples, simulating application of the proposed programming changes can include simulating application of the proposed programming change (or a plurality of programming changes including the proposed programming change) on the digital model of the ambulatory medical device and the digital model of the patient to predict the outcome of the proposed programming change based on training data from a plurality of other ambulatory medical devices and a plurality of other patients.

In an example, updating the digital model at step 303 can include updating the digital model of the ambulatory medical device based on the predicted outcome of the proposed programming change, updating the digital model of the patient based on the predicted outcome of the proposed programming change, or combinations thereof.

At step 305, one or more of the determined changes can be compared to a threshold, such as using an assessment circuit or one or more other components. In certain examples, the threshold can include a patient-specific or clinical threshold, a relative threshold determined as a percentage change from a baseline or one or more other values or parameters, or one or more other thresholds described herein, etc., such as a relative threshold based on a patient response metric in one or more previous time periods, a patient baseline, or an expected positive or negative trend of a patient response metric over a time period of a respective therapy, parameter setting, or combination of parameter settings, such as with respect to a previous time period or an expected value, for example, selected or determined by a clinician or with reference to the patient or a group of patients. In certain examples, at least one of the one or more thresholds can include alert thresholds indicative of a worsening patient status requiring clinician review. In other examples, the one or more thresholds can be indicative of a worsening patient status, such as to trigger a therapy or parameter setting adjustment, but below the alert threshold.

In an example, comparison to the one or more thresholds can occur after a period to time, and in certain examples a report can be determined based on the determined patient response metric for the period of time. In other examples, comparison to the one or more thresholds can occur in real-time, irrespective of time periods, with an alert provided if the determined patient response metric exceeds the threshold.

If the one or more determined changes, alone or in combination, do not exceed the threshold, indicating a stable or improving patient status or condition or a stable or improving ambulatory medical device performance, the process can return to one or more previous steps, such as step 301 or step 302. If the one or more determined changes, alone or in combination, exceed the threshold, indicating a worsening patient status or condition with respect to the threshold or a worsening ambulatory medical device condition or performance, the method can proceed to step 306.

At step 306, a programming recommendation can be generated, such as using an assessment circuit or one or more other components, in response to the determined changes in the one or more digital models to optimize resources of the ambulatory medical device. In an example, the programming recommendation can be based on the predicted change in the one or more updated digital models, such as the digital model of the patient, the digital model of the ambulatory medical device, or combination thereof. In other examples, the programming recommendation can be based on the predicted outcome of the proposed programming change.

In an example, the programming recommendation can include a recommendation to change at least one of a sensing, pacing, storage, communication, or therapy parameter of the ambulatory medical device to optimize resources of the ambulatory medical device. The programming recommendation can be provided to a user or can trigger application of the determined changes, in combination with the one or more digital models, to one or more pre-trained machine learning models, such as by one or more assessment circuits of a patient management system, including, for example, one or more remote devices, etc.

In an example, such as in response to a received proposed change at step 302, the programming recommendation can include a comprehensive evaluation of the likely success of the received proposed change at step 302, potentially including a percent confidence, risk assessment, and indications of potential concerns or issues, a recommendation to update existing parameter settings or therapy to the received proposed change at step 302, or combinations thereof.

In an example, an alert can be provided, such as by the assessment circuit, for example, if the one or more determined changes exceeds the one or more thresholds, or in other examples if one or more reports of determined changes are available for review or transmission, if one or more changes are determined or detected, such as above an alert threshold, etc. In an example, an output can be provided of the alert to a user interface for display to a user or to another circuit to control or adjust a process or a function of the ambulatory medical device, such as to adjust a therapy, mode, parameter settings, or follow-up schedule associated with the patient, a clinician, etc., with each of such adjustments at least partially dependent on the value of the determined change or the amount that the determined change exceeds the one or more thresholds. In an example, the alert can include recommended updated parameter settings for clinician review, and the clinician can instruct updated parameter settings to be applied responsive thereto.

At step 307, updated parameter settings or therapy modifications can be programmed to the ambulatory medical device or otherwise implemented by the assessment circuit or one or more other components, through one or more communication circuits, etc., to optimize resources of the ambulatory medical device. In an example, an output of the generated programming recommendation, such as including parameter settings, a proposed ambulatory medical device or therapy, etc., to a user interface for display to the user or to a control circuit to control or adjust the process or function of a medical device system, etc. The generated programming recommendation settings can be stored, such as using the assessment circuit, and transmitted, by control of the assessment circuit or using one or more communication circuits, etc., such as to one or more additional processes or components, such as an output circuit (e.g., a display, a controller for a display, etc.). In certain examples, the generated programming recommendation can be programmed to the ambulatory medical device without requiring an in-clinic follow-up appointment by the patient.

Implementation of the steps above can, in certain examples, result in a two-factor approval process for accepting and applying received programming changes, in that the received programming changes have physician-level approval, such as in response to receiving the proposed change or after providing results of the determined change in the one or more models being presented to a clinician or other user, as well as approval by the determined changes in the one or more models, or separately, one or more other users, before the proposed changes are applied to the ambulatory medical device. Such steps allow for cloud-based evaluation and prediction of programming changes before they are implemented on the ambulatory medical device, improving safety, efficacy, and efficiency of ambulatory medical device management.

Once the updated parameter settings are applied to the ambulatory medical device, process can return to step 301 for evaluation with respect to the one or more digital models. In an example, if the one or more digital models indicate that the patient or ambulatory medical device are stable or improving (e.g., changes do not exceed the one or more thresholds at step 305, etc.), a clinician follow-up (e.g., an in-person follow-up) may not yet be necessary (e.g., additional parameter settings can be tried, an alert can be provided to a clinician for review, etc.). In other examples, if the one or more digital models indicate that the patient or ambulatory medical device are worsening (e.g., changes do not exceed the one or more thresholds at step 305, etc.), a follow-up can be scheduled or an existing follow-up schedule can be modified (e.g., to occur sooner, etc.), such as by one or more circuits (e.g., an assessment circuit, a scheduling circuit, etc.) or components of an external system (e.g., an external device, a remote device, etc.), etc.

In other examples, separate from clinician follow-up with the patient, one or more other conditions can be adjusted to optimize resources of the ambulatory medical device, including, for example, sampling, storage, or communication of physiologic information of the patient or device information frequency of communication by one or more communication circuits of the ambulatory medical device. If the determined changes in the one or more models are stable, improving, or behaving as expected (such as predicted by the one or more models), ambulatory medical device resources can be adjusted to improve efficiency, such as by reducing sampling frequency of one or more sensors, reduce data processing, storage, or transmission of information outside of the ambulatory medical device, optimizing resources of the ambulatory medical device. When using digital models that are further influenced by one or more other ambulatory medical devices or physiologic information from one or more other patients, stability in patient-specific signals alone can be expanded to expected performance with greater sensitivity and specificity, further improving device performance in contrast to using patient information alone, such use of the one or more digital models additionally improves device management. Further, use of models can simulate device status without directly polling the device, such as when cloud-based simulation is desired or when improvements are identified by other users separate from the clinician of the patient or outside of a follow-up setting.

Similarly, when the one or more digital models are working as expected, such that subsequent information from the patient or the ambulatory medical device substantially agrees with predicted changes (e.g., within a threshold of an expected amount, etc.), such as output of simulations or predictions prior to implementing one or more changes, etc., existing training data for the one or more models is confirmed such that the subsequent data from the patient or the ambulatory medical device that confirms the existing training data does not need to be processed into the training data, reducing the amount of processing required by the machine learning models. Such concept additionally applies to transmission of data out of the ambulatory medical device. If the data sensed or received by the ambulatory medical device agrees with a predicted response (e.g., within a threshold of an expected amount, etc.), such data does not have to be stored or transmitted, reducing resource usage and optimizing resources of the ambulatory medical device.

In contrast, when subsequent information disagrees with predicted information from the one or more models, such as when simulations of proposed programming changes do not agree (e.g., exceeds a threshold of an expected amount, etc.) with subsequent information from the patient or the ambulatory medical device, the additional data from the patient or the ambulatory medical device that disagrees with the existing training data may need to be processed into the training data, updating the machine learning model. However, subsequent updates of the training data will lead to more efficient subsequent training of the ambulatory medical device, optimizing resources of the ambulatory medical device, as well as optimizing resources of other ambulatory medical devices for the patient or other patients.

At step 308, consistent with above, training data can be updated, such as using an assessment circuit or one or more other components. For example, at step 303, application of a proposed programming change received at step 302 can be simulated on the one or more digital models and a predicted change in the one or more digital models of the proposed programming change can be determined based on training data. Subsequent to implementation of a programming change, such as the proposed programming change or one or more other generated programming recommendations, additional physiologic information can be received representative of the programming change. The one or more digital models can be updated based on the additional physiologic information and a determination of a difference between the one or more updated digital models and the predicted change. If the determined difference is below an update threshold, indicating that the simulation or predicted change agrees with the resulting outcome, existing training data can be maintained (e.g., the training data is not updated). However, if the determined difference exceeds the update threshold, indicating that the simulation or predicted change disagrees with the resulting outcome, existing training data can be updated to account for or include at least one of the programming change, the resulting outcome (e.g., physiologic information of the patient), or the simulation or predicted change. Determining that the existing training data should be updated comprises additional processing and in certain examples communication of parameter settings and physiologic information for subsequent training. Whereas determining to not update the training data is a direct reduction in resources such as in contrast to updating the training data, determining to update the training data in situations where the one or more models are determined as insufficient in some way, such as simulation or predicted change, etc., improves subsequent simulation and predicted changes and the speed at which programming changes arrive at optimal programming, optimizing resources of the ambulatory medical device.

Although illustrated as a series of steps above, in certain examples, one or more steps are optional, and in other examples, different combinations or permutations of these or other steps or examples can be combined to form other methods or processes, which is also applicable to other examples discussed herein.

### Medical Device System

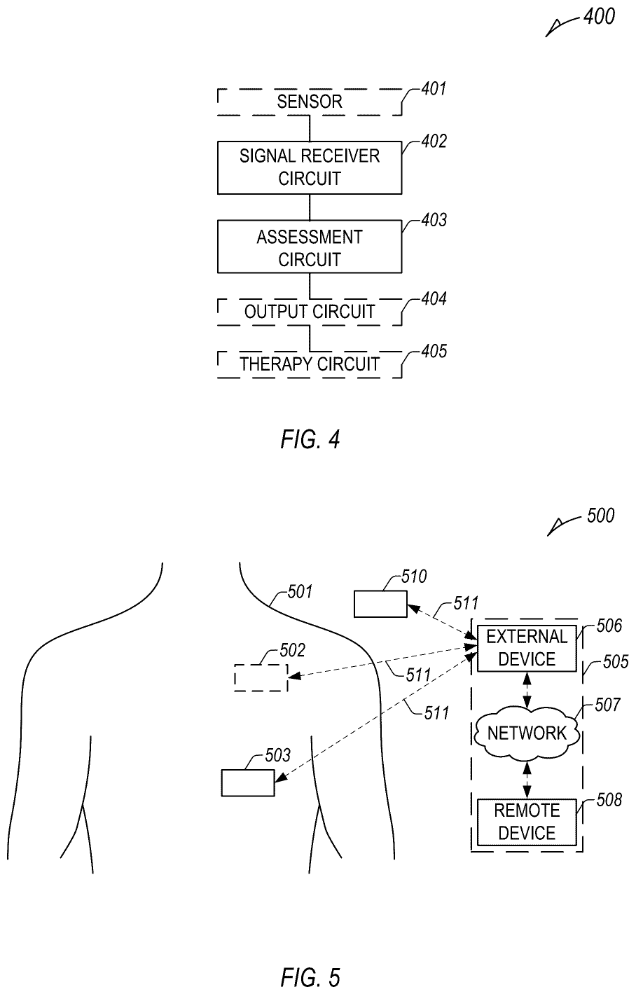

FIG. 4 illustrates an example system 400 (e.g., a medical device system). In an example, one or more aspects of the system 400 can be a component of, or communicatively coupled to, a medical device, such as an implantable medical device (IMD), an insertable cardiac monitor, an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a body of a patient or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 402 can include the sensor 401. In other examples, the signal receiver circuit can be coupled to or a component of the assessment circuit 403.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

### Efficiency and Mode Transitions

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiratory rate (RR), tidal volume (TV), rapid shallow breathing index (RSBI), respiration phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Physiologic Parameters

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (Si through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (Si) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiratory rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiratory rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

### Alert States

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Patient Indications

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is the HeartLogic™ index, a HeartLogic™ in-alert time, or one or more other composite measurements or measures thereof. The HeartLogic™ index is a composite indication of patient condition determined using different combinations or weightings of physiologic information, including two or more of Si heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

### Patient Management System

FIG. 5 illustrates an example patient management system 500 and portions of an environment in which the patient management system 500 may operate. The patient management system 500 can perform a range of activities, including remote patient monitoring and diagnosis of a disease condition, programming of ambulatory medical devices, and control of one or more therapies. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device, such as an implantable medical device 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 502 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include cardiac resynchronization therapy for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming or reprogramming the implantable medical device 502 with different parameter settings to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The remote device 508 can be configured to evaluate collected device or patient information and provide alert notifications, among other possible functions. In an example, the remote device 508 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 505 or one or more components thereof (e.g., the external device 506, the remote device 508, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 506 or the remote device 508 can include a respective display unit for displaying the physiologic or functional signals, or alerts, alarms, emergency calls, or other forms of warnings to signal the detection of one or more conditions. In some examples, the external system 505 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 502, the wearable medical device 503, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 508, or via a user interface of a software application executing on a client device communicatively connected with the remote device 508. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 505 can be implemented using hardware, software, firmware, or combinations thereof. Portions of the one or more ambulatory medical devices or the external system 505 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming or reprogramming the one or more ambulatory medical devices and can receive information about one or more signals acquired by the one or more ambulatory medical devices, such as can be received via a communication link 511. The external system 505 can include a local external implantable medical device programmer. The external system 505 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (Si) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia event, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

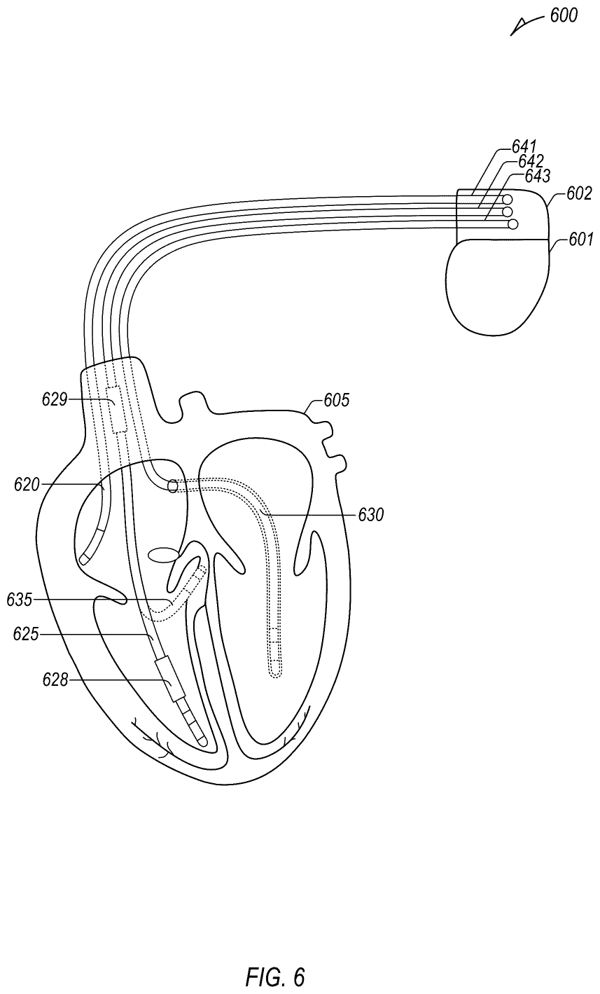

FIG. 6 illustrates an example implantable medical device system 600 including an implantable medical device 601 electrically coupled to a heart 605, such as through one or more leads coupled to the implantable medical device 601 through one or more lead ports, including first, second, or third lead ports 641, 642, 643 in a header 602 of the implantable medical device 601. In an example, the implantable medical device 601 can include an antenna, such as in the header 602, configured to enable communication with an external system and one or more electronic circuits (e.g., an assessment circuit, etc.) in a hermetically sealed housing (CAN).

The implantable medical device 601 may include an insertable cardiac monitor, pacemaker, defibrillator, cardiac resynchronization therapy device, or other subcutaneous implantable medical device or cardiac rhythm management (CRM) device configured to be implanted in a chest of a subject, having one or more leads to position one or more electrodes or other sensors at various locations in or near the heart 605, such as in one or more of the atria or ventricles. Separate from, or in addition to, the one or more electrodes or other sensors of the leads, the implantable medical device system 600 can include one or more electrodes or other sensors (e.g., a pressure sensor, an accelerometer, a gyroscope, a microphone, etc.) powered by a power source in the implantable medical device 601. The one or more electrodes or other sensors of the leads, the implantable medical device 601, or a combination thereof, can be configured to detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 605 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 601 can include one or more electronic circuits configured to sense one or more physiologic signals, such as an electrogram or a signal representing mechanical function of the heart 605. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 628, the second defibrillation coil electrode 629, etc.) may be used together with the housing to deliver one or more cardioversion/defibrillation pulses.

In an example, the implantable medical device 601 can sense impedance such as between electrodes located on one or more of the leads or the housing. The implantable medical device 601 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 601 can be configured to inject current between an electrode on one or more of the first, second, third, or fourth leads 620, 625, 630, 635 and the housing, and to sense the resultant voltage between the same or different electrodes and the housing.

The implantable medical device 601 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are.

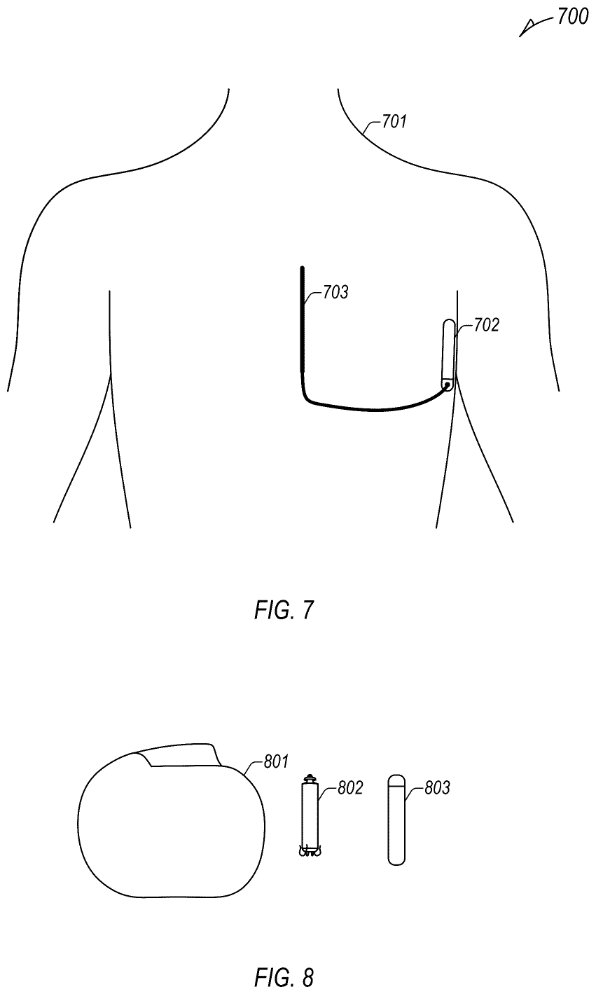

FIGS. 7-8 illustrate example ambulatory medical devices systems, devices, and remote patient or programming systems. FIG. 7 illustrates an example implantable medical device system 700 including a subcutaneous implantable cardioverter defibrillator 702 implanted at a lateral thoracic region of a patient 701 and a subcutaneous lead 703 including a defibrillation coil and proximal and distal sensing electrodes implanted subcutaneously along a thorax of the patient 701. FIG. 8 illustrates example implantable medical devices, including a subcutaneous implantable cardioverter defibrillator 801, a leadless cardiac pacemaker 802 including tines for placement, and an insertable cardiac monitor 803.

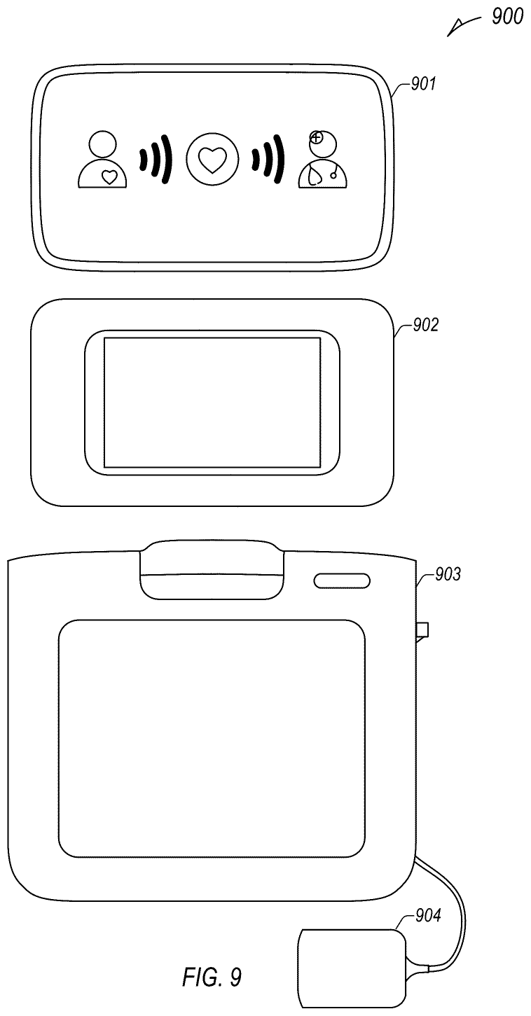

FIG. 9 illustrates example remote patient management systems 900 for communication with an ambulatory medical device, such as for receiving information from or providing information to, including programming instructions, one or more ambulatory medical devices. The example remote patient management systems 900 include a first remote patient management device 901 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 902 (e.g., an EMBLEM™ S-ICD Programmer) with RF telemetry capabilities through one or more communication circuits and an optional external telemetry wand for communication with an ambulatory medical device, and a third remote patient management system 903 (e.g., a LATITUDE™ Programming System, Model 3300, etc.) with RF telemetry capabilities through one or more communication circuits and an external telemetry wand 904 (e.g., a Model 6395 Telemetry Wand, etc.) including an external telemetry coil configured for inductive communication with a corresponding telemetry coil of an implantable medical device. Although not illustrated herein, the remote patient management systems 900 can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

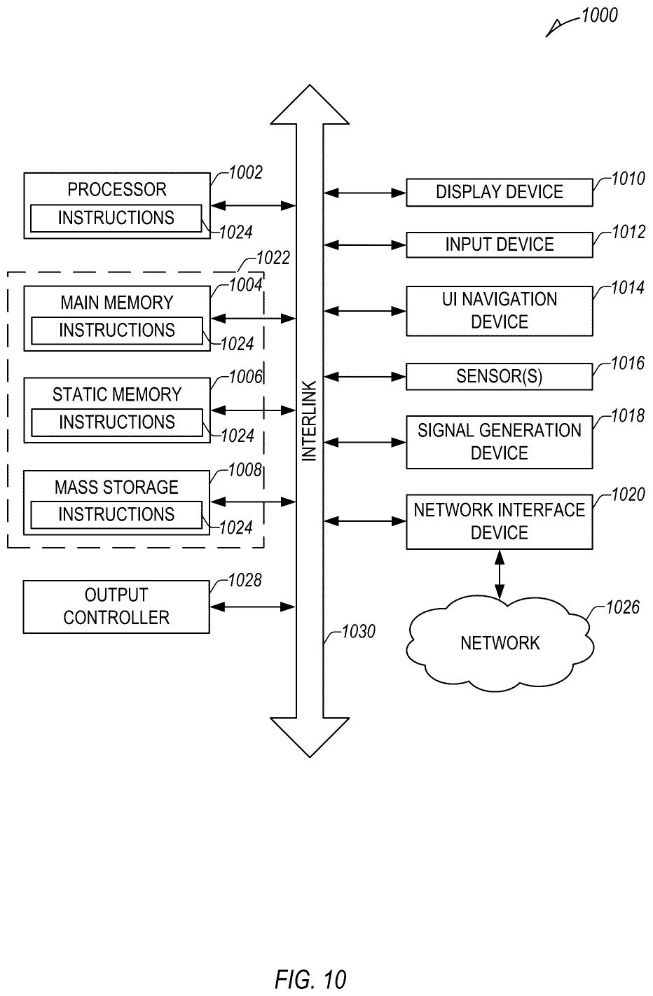

FIG. 10 illustrates a block diagram of an example machine 1000 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1000. Circuitry (e.g., processing circuitry, an assessment circuit, etc.) is a collection of circuits implemented in tangible entities of the machine 1000 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1000 follow.

In alternative embodiments, the machine 1000 may operate as a standalone device or may be connected (e.g., networked) to other machines. In a networked deployment, the machine 1000 may operate in the capacity of a server machine, a client machine, or both in server-client network environments. In an example, the machine 1000 may function as a peer machine in peer-to-peer (P2P) (or other distributed) network environment. The machine 1000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1000 (e.g., computer system) may include a hardware processor 1002 (e.g., a central processing unit (CPU), a graphics processing unit (GPU), a hardware processor core, or any combination thereof), a main memory 1004, a static memory 1006 (e.g., memory or storage for firmware, microcode, a basic-input-output (BIOS), unified extensible firmware interface (UEFI), etc.), and mass storage 1008 (e.g., hard drive, tape drive, flash storage, or other block devices) some or all of which may communicate with each other via an interlink 1030 (e.g., bus). The machine 1000 may further include a display unit 1010, an alphanumeric input device 1012 (e.g., a keyboard), and a user interface (UI) navigation device 1014 (e.g., a mouse). In an example, the display unit 1010, input device 1012, and UI navigation device 1014 may be a touch screen display. The machine 1000 may additionally include a signal generation device 1018 (e.g., a speaker), a network interface device 1020, and one or more sensors 1016, such as a global positioning system (GPS) sensor, compass, accelerometer, or one or more other sensors. The machine 1000 may include an output controller 1028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1002, the main memory 1004, the static memory 1006, or the mass storage 1008 may be, or include, a machine-readable medium 1022 on which is stored one or more sets of data structures or instructions 1024 (e.g., software) embodying or utilized by any one or more of the techniques or functions described herein. The instructions 1024 may also reside, completely or at least partially, within any of registers of the processor 1002, the main memory 1004, the static memory 1006, or the mass storage 1008 during execution thereof by the machine 1000. In an example, one or any combination of the hardware processor 1002, the main memory 1004, the static memory 1006, or the mass storage 1008 may constitute the machine-readable medium 1022. While the machine-readable medium 1022 is illustrated as a single medium, the term “machine-readable medium” may include a single medium or multiple media (e.g., a centralized or distributed database, and/or associated caches and servers) configured to store the one or more instructions 1024.

The term “machine-readable medium” may include any medium that is capable of storing, encoding, or carrying instructions for execution by the machine 1000 and that cause the machine 1000 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1024 may be further transmitted or received over a communications network 1026 using a transmission medium via the network interface device 1020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1020 may include one or more physical jacks (e.g., Ethernet, coaxial, or phone jacks) or one or more antennas to connect to the communications network 1026. In an example, the network interface device 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1000, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for evaluating patient condition and programming an ambulatory medical device to optimize resources of the ambulatory medical device, the system comprising:
one or more processors; and
one or more memory devices storing instructions, which when executed by the one or more processor, cause the one or more processors to perform operations comprising:
receiving physiologic information of a patient from the ambulatory medical device, including physiologic information of the patient in a first time period;
generating a digital model of the patient based on the received physiologic information of the patient in the first time period;
receiving additional physiologic information of the patient from the ambulatory medical device subsequent to the first time period;
updating the digital model of the patient based on the received additional physiologic information of the patient subsequent to the first time period;
storing the updated digital model of the patient for use in evaluating programming changes for the ambulatory medical device;
determining a change in the updated digital model of the patient;
generating, based on the determined change in the updated digital model of the patient, a programming recommendation for the ambulatory medical device to optimize resources of the ambulatory medical device; and
programming the ambulatory medical device based on the generated programming recommendation to optimize resources of the ambulatory medical device.

2. The system of claim 1, wherein the ambulatory medical device comprises an implantable medical device,
wherein the operations comprise receiving an indication of the time of implant of the implantable medical device,
wherein the first time period comprises a post-implant time period including a pre-determined time period from the time of implant of the implantable medical device.

3. The system of claim 1, wherein the ambulatory medical device of the patient is a first type of device,
wherein the operations comprise receiving physiologic information from a plurality of other patients have the first type of ambulatory medical device,
wherein updating the digital model of the patient comprises continuously updating the digital model of the patient based on the received physiologic information from the plurality of other patients.

4. The system of claim 1, wherein the operations comprise receiving parameter settings of the ambulatory medical device in the first time period,
wherein generating the digital model of the patient comprises based on the received physiologic information of the patient and the received parameter settings of the ambulatory medical device in first time period.

5. The system of claim 1, wherein updating the digital model of the patient comprises continuously updating the digital model of the patient based on parameter settings of the ambulatory medical device.

6. The system of claim 1, wherein generating the programming recommendation for the ambulatory medical device comprises generating a reprogramming recommendation to change at least one of a sensing, pacing, storage, communication, or therapy parameter of the ambulatory medical device to optimize resources of the ambulatory medical device.

7. The system of claim 1, wherein the operations comprise:
generating, based on the generated programming recommendation, an alert to a user or process and providing an indication of the generated programming recommendation to the user or process; and
receiving an instruction to program the generated programming recommendation,
wherein programming the ambulatory medical device based on the generated programming recommendation comprises programming the ambulatory medical device to implement the generated programming recommendation based on the received instruction to program the generated programming recommendation.

8. The system of claim 1, wherein the operations comprise:
generating or accessing a digital model of the ambulatory medical device based on parameter settings of the ambulatory medical device; and
receiving a proposed programming change for the ambulatory medical device, the proposed programming change including at least one of a therapy change or a parameter setting change,
wherein determining the change in the updated digital model of the patient comprises simulating application of the proposed programming change on the digital model of the patient using the digital model of the ambulatory medical device and predicting a change in the updated digital model of the patient of the proposed programming change based on training data from a plurality of other patients or ambulatory medical devices,
wherein generating the programming recommendation for the ambulatory medical device comprises based on the predicted change in the updated digital model of the patient.

9. The system of claim 1, wherein programming the ambulatory medical device based on the generated programming recommendation comprises programming the ambulatory medical device to implement the generated programming recommendation.

10. The system of claim 1, wherein
the operations further comprise determining a difference between the additional physiologic information subsequent to the first time period and the physiologic information from the first time period, wherein updating the digital model of the patient comprises updating the digital model of the patient based on the determined difference between the additional physiologic information subsequent to the first time period and the physiologic information from the first time period.

11. The system of claim 10, wherein the operations comprise:
generating a digital model of the ambulatory medical device based on received parameter settings of the ambulatory medical device; and
simulating application of the programming recommendation on both the digital model of the patient and the digital model of the ambulatory medical device to predict an outcome of the programming recommendation before programming the ambulatory medical device.

12. The system of claim 10, wherein programming the ambulatory medical device to optimize resources of the ambulatory medical device comprises adjusting at least one of a sampling frequency of sensors, a data processing rate, a data storage rate, or a data transmission rate of the ambulatory medical device based on the determined changes in the continuously updated digital model of the patient.

13. A system for predicting an outcome of programming changes to an ambulatory medical device and programming the ambulatory medical device to optimize resources of the ambulatory medical device, the system comprising:
one or more processors; and
one or more memory devices storing instructions, which when executed by the one or more processor, cause the one or more processors to perform operations comprising:
receiving parameter settings of the ambulatory medical device;
generating a digital model of the ambulatory medical device based on the received parameter settings of the ambulatory medical device;
receiving a proposed programming change for the ambulatory medical device, the proposed programming change including at least one of a therapy change or a parameter setting change;
updating the digital model of the ambulatory medical device based on the proposed programming change, including simulating application of the proposed programming change on the digital model of the ambulatory medical device to predict an outcome of the proposed programming change based on training data from a plurality of other ambulatory medical devices;
determining a change in the updated digital model of the ambulatory medical device;
generating, based on the determined change in the updated digital model of the ambulatory medical device, a programming recommendation for the ambulatory medical device to optimize resources of the ambulatory medical device; and
programming the ambulatory medical device based on the generated programming recommendation to optimize resources of the ambulatory medical device.

14. The system of claim 13, wherein the ambulatory medical device is a first type of device,
wherein the plurality of other ambulatory medical devices are the first type of device,
wherein the operations comprise updating the digital model of the ambulatory medical device based on the received physiologic information from the plurality of other ambulatory medical devices.

15. The system of claim 13, wherein the operations comprise:
generating or accessing a digital model of the patient based on physiologic information of the patient received from the ambulatory medical device; and
receiving a proposed programming change for the ambulatory medical device, the proposed programming change including at least one of a therapy change or a parameter setting change,
wherein simulating application of the proposed programming change comprises simulating application of the proposed programming change on the digital model of the ambulatory medical device and the digital model of the patient to predict the outcome of the proposed programming change based on training data from a plurality of other ambulatory medical devices and a plurality of other patients,
wherein updating the digital model of the ambulatory medical device comprises updating the digital model of the ambulatory medical device based on the predicted outcome of the proposed programming change,
wherein the operations comprise updating the digital model of the patient based on the predicted outcome of the proposed programming change,
wherein generating the programming recommendation for the ambulatory medical device comprises based on the predicted outcome of the proposed programming change.

16. The system of claim 15, wherein simulating application of the proposed programming change comprises simulating application of a plurality of programming changes including the proposed programming change and predicting the outcome of the plurality of programming changes including the proposed programming change based on training data from a plurality of other patients or the training data from the plurality of other ambulatory medical devices.

17. The system of claim 13, wherein determining the change in the updated digital model of the ambulatory medical device based on the predicted outcome of the proposed programming change.

18. A system for evaluating patient condition and programming an ambulatory medical device to optimize resources of the ambulatory medical device, the system comprising:
one or more processors; and
one or more memory devices storing instructions, which when executed by the one or more processor, cause the one or more processors to perform operations comprising:
receiving physiologic information of a patient from the ambulatory medical device;
continuously updating a stored digital model of the patient based on the received physiologic information of the patient;
determining changes in the continuously updated digital model of the patient;
generating, based on the determined changes in the continuously updated digital model of the patient, a programming recommendation for the ambulatory medical device to optimize resources of the ambulatory medical device; and
programming the ambulatory medical device based on the generated programming recommendation to optimize resources of the ambulatory medical device.

19. The system of claim 18, wherein the operations comprise:
receiving information of the ambulatory medical device;
continuously updating a stored digital model of the ambulatory medical device based on the received information of the ambulatory medical device;
determining changes in the continuously updated digital model of the ambulatory medical device,
wherein generating the programming recommendation includes based on the determined changes in the continuously updated digital model of the patient and the determined changes in the continuously updated digital model of the ambulatory medical device.

20. The system of claim 18, wherein programming the ambulatory medical device to optimize resources of the ambulatory medical device comprises adjusting at least one of a sampling frequency of sensors, a data processing rate, a data storage rate, or a data transmission rate of the ambulatory medical device based on the determined changes in the continuously updated digital model of the patient.
